SEQUENCE LISTING (50 sequences)

SEQ ID NO: 1 - DNA, 20 bp, Artificial Sequence
  Description of Artificial Sequence Synthetic primer
gatacgtccagtcacacttc

SEQ ID NO: 2 - DNA, 21 bp, Artificial Sequence
  Description of Artificial Sequence Synthetic primer
gttcctgcactgtgtaaagtg

SEQ ID NO: 3 - DNA, 23 bp, Artificial Sequence
  Description of Artificial Sequence Synthetic primer
gcaatcgactccaaatcaaactc

SEQ ID NO: 4 - DNA, 20 bp, Artificial Sequence
  Description of Artificial Sequence Synthetic primer
gtgacaagcacccagatctg

SEQ ID NO: 5 - DNA, 20 bp, Artificial Sequence
  Description of Artificial Sequence Synthetic primer
ctgatatggcagcacagaag

SEQ ID NO: 6 - DNA, 21 bp, Artificial Sequence
  Description of Artificial Sequence Synthetic primer
gagcagctaatacgaaggatc

SEQ ID NO: 7 - DNA, 21 bp, Artificial Sequence
  Description of Artificial Sequence Synthetic primer
gcactagtaaccctcaatctc

SEQ ID NO: 8 - DNA, 22 bp, Artificial Sequence
  Description of Artificial Sequence Synthetic primer
ccttcaattgcatcataggtac

SEQ ID NO: 9 - DNA, 21 bp, Artificial Sequence
  Description of Artificial Sequence Synthetic primer
ctgttcacagcgcataagttc

SEQ ID NO: 10 - DNA, 22 bp, Artificial Sequence
  Description of Artificial Sequence Synthetic primer
gaatggaattcaagtggacatc

SEQ ID NO: 11 - DNA, 21 bp, Artificial Sequence
  Description of Artificial Sequence Synthetic primer
ctctgtcaactctgtatggag

SEQ ID NO: 12 - DNA, 22 bp, Artificial Sequence
  Description of Artificial Sequence Synthetic primer
ctgttctatttccctaatgctc

SEQ ID NO: 13 - DNA, 20 bp, Artificial Sequence
  Description of Artificial Sequence Synthetic primer
cagtcgttacagaggaacag

SEQ ID NO: 14 - DNA, 21 bp, Artificial Sequence
  Description of Artificial Sequence Synthetic primer
gcattcgttgctgcatcgttg

SEQ ID NO: 15 - DNA, 21 bp, Artificial Sequence
  Description of Artificial Sequence Synthetic primer
ggaagaccgattgctttagac

SEQ ID NO: 16 - DNA, 20 bp, Artificial Sequence
  Description of Artificial Sequence Synthetic primer
gagaatgcatcgtcgtgttc

SEQ ID NO: 17 - DNA, 22 bp, Artificial Sequence
  Description of Artificial Sequence Synthetic primer
attgacgggagtgtattgacgc

SEQ ID NO: 18 - DNA, 21 bp, Artificial Sequence
  Description of Artificial Sequence Synthetic primer
cataagtgtgccttagtatgc

SEQ ID NO: 19 - DNA, 21 bp, Artificial Sequence
  Description of Artificial Sequence Synthetic primer
catttcccaagaggatcaatc

SEQ ID NO: 20 - DNA, 21 bp, Artificial Sequence
  Description of Artificial Sequence Synthetic primer
ccaacagatataggctgtgtc

SEQ ID NO: 21 - DNA, 21 bp, Artificial Sequence
  Description of Artificial Sequence Synthetic primer
ctatgctgtctcaccatagag

SEQ ID NO: 22 - DNA, 20 bp, Artificial Sequence
  Description of Artificial Sequence Synthetic primer
ccatcaagcccattcaatgc

SEQ ID NO: 23 - DNA, 22 bp, Artificial Sequence
  Description of Artificial Sequence Synthetic primer
cgatggaagaggtaaagtagtc

SEQ ID NO: 24 - DNA, 21 bp, Artificial Sequence
  Description of Artificial Sequence Synthetic primer
gtactggtgaaagaatgtctg

SEQ ID NO: 25 - DNA, 20 bp, Artificial Sequence
  Description of Artificial Sequence Synthetic primer
ctccaacaattacacatcca

SEQ ID NO: 26 - DNA, 21 bp, Artificial Sequence
  Description of Artificial Sequence Synthetic primer
ttctctccgccaggaaactga

SEQ ID NO: 27 - DNA, 21 bp, Artificial Sequence
  Description of Artificial Sequence Synthetic primer
cgacaaagtatctcagaaggg

SEQ ID NO: 28 - DNA, 22 bp, Artificial Sequence
  Description of Artificial Sequence Synthetic primer
gaagtgcagagttatctgctac

SEQ ID NO: 29 - DNA, 23 bp, Artificial Sequence
  Description of Artificial Sequence Synthetic primer
gaaggttcatattctgtgaacgc

SEQ ID NO: 30 - DNA, 21 bp, Artificial Sequence
  Description of Artificial Sequence Synthetic primer
cttgtagctgttgagctgtac

SEQ ID NO: 31 - DNA, 20 bp, Artificial Sequence
  Description of Artificial Sequence Synthetic primer
gaaactgatggactccgtag

SEQ ID NO: 32 - DNA, 21 bp, Artificial Sequence
  Description of Artificial Sequence Synthetic primer
gtacatccggattttcagaag

SEQ ID NO: 33 - DNA, 21 bp, Artificial Sequence
  Description of Artificial Sequence Synthetic primer
ctgatccagaaaaggcaagag

SEQ ID NO: 34 - DNA, 21 bp, Artificial Sequence
  Description of Artificial Sequence Synthetic primer
ccgcctagtgcttaagagttc

SEQ ID NO: 35 - DNA, 21 bp, Artificial Sequence
  Description of Artificial Sequence Synthetic primer
gatctgtatatcgtcgtcacc

SEQ ID NO: 36 - DNA, 20 bp, Artificial Sequence
  Description of Artificial Sequence Synthetic primer
cttccaccagtaaacgtttc

SEQ ID NO: 37 - DNA, 20 bp, Artificial Sequence
  Description of Artificial Sequence Synthetic primer
cctctttatatgatctgccg

SEQ ID NO: 38 - DNA, 20 bp, Artificial Sequence
  Description of Artificial Sequence Synthetic primer
gttgaaacgccaacagtggc

SEQ ID NO: 39 - DNA, 21 bp, Artificial Sequence
  Description of Artificial Sequence Synthetic primer
catcgtaaacgtgtgtagagc

SEQ ID NO: 40 - DNA, 20 bp, Artificial Sequence
  Description of Artificial Sequence Synthetic primer
cgatctatagggactatcgg

SEQ ID NO: 41 - DNA, 22 bp, Artificial Sequence
  Description of Artificial Sequence Synthetic primer
ctaagttcggaacacaaaaccc

SEQ ID NO: 42 - DNA, 24 bp, Artificial Sequence
  Description of Artificial Sequence Synthetic primer
tctcgcttgtctatccaaaccttc

SEQ ID NO: 43 - DNA, 20 bp, Artificial Sequence
  Description of Artificial Sequence Synthetic primer
ctcgttctcaacaactctac

SEQ ID NO: 44 - DNA, 24 bp, Artificial Sequence
  Description of Artificial Sequence Synthetic primer
cctaatacctcaatggtccaatac

SEQ ID NO: 45 - DNA, 21 bp, Artificial Sequence
  Description of Artificial Sequence Synthetic primer
gcagatagaccttctacatgg

SEQ ID NO: 46 - DNA, 21 bp, Artificial Sequence
  Description of Artificial Sequence Synthetic primer
ctaaggtagccagaacttctc

SEQ ID NO: 47 - DNA, 19 bp, Artificial Sequence
  Description of Artificial Sequence Synthetic primer
gctgcacgtgactaactag

SEQ ID NO: 48 - DNA, 23 bp, Artificial Sequence
  Description of Artificial Sequence Synthetic primer
catcataagagcataagcgacag

SEQ ID NO: 49 - DNA, 20 bp, Artificial Sequence
  Description of Artificial Sequence Synthetic primer
gccgatttcgctttagaacc

SEQ ID NO: 50 - DNA, 2205 bp, Artificial Sequence
  Description of Artificial Sequence Synthetic nucleotide construct
agatcttgagctcgactgcagctcggagacaatcatatgggagaagcaattggaagatagaaaaaaggtactcggtacataaatatatgtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactcacttcaacttcatcttacaaaagatcacgtgatctgttgtattgaactgaaaattttttgtttgcttctctctctctctttcattatgtgagagtttaaaaaccagaaactacatcatcgaaaaagagtttaaaccattacaaccattgcgataagccctctcaaacttcctccagactgtttgtcatccaattggtaagattaattatcatataccccggataagaaaagaagcaagcacacaatggatcaactggccggcccgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatctcatatgcctgcagg

(19) United States
(12) Patent Application Publication
(10) Pub. No.: US 2006/0105361 A1
(43) Pub. Date: May 18, 2006

(54) Donor yeast strain for transfer of genetic material

(76) Inventors: Rodney J. Rothstein, Maplewood, NJ (US); Robert J.D. Reid, New York, NY (US); Ivana Sunjevaric, New York, NY (US); Marisa Wagner, New York, NY (US); Samantha Ciccone, Brooklyn, NY (US)

Correspondence Address: WILMER CUTLER PICKERING HALE AND DORR LLP, 399 PARK AVENUE, NEW YORK, NY, 10022, US

(21) Appl. No.: 11/211,359

(22) Filed: Aug. 24, 2005

Related U.S. Application Data
(60) Provisional application No. 60/603,889, filed on Aug. 24, 2004.

(51) Int. Cl. C12Q 1/68; C12N 15/74; C12N 1/18

(52) U.S. Cl. 435/6; 435/483; 435/254.2

(57) ABSTRACT

The invention provides a universal yeast donor strain that contains a conditional centromere and a URA3 allele on every chromosome. This strain was constructed in four rounds of crosses of individual conditional chromosome strains using a novel tetrad-based screen to identify segregants in which all marked chromosomes were contained in the same spore. The invention also provides an improved high efficiency method to transfer extrachromosomal genetic material such as plasmid DNA into any Saccharomyces strain for use with the current gene disruption libraries. The method of transfer is mating-based method which uses a kar1 plasmid donor strain that can initiate mating but cannot form a diploid and allows plasmid transfer (plasmoduction) between nuclei in the heterokaryon. kar1 matings have been used to transfer YACs between yeast strains, but previous methods required specialized genetic backgrounds in the recipient strains and suffered from high rates of spurious chromosome transfer (Hugerat, Y., et al. 1994. Genomics 22:108). Plasmoduction with the universal donor strain only requires that the recipient strain be ura3, GAL+ and have another marker available for selection of the transferred plasmid. Counterselection against every donor chromosome also limits the amount of spurious allele transfer. The universal donor strain and the method of the invention are used to screen the yeast gene disruption library with plasmid-based dominant negative alleles of various genes.

This application claims the benefit of priority of U.S. Provisional Patent Application Ser. No. 60/603,889, filed Aug. 24, 2004, which is hereby incorporated by reference in its entirety for all purposes.

The invention disclosed herein was made with U.S. Government support from National Institute of Health Grant R21 HG002614. Accordingly, the U.S. Government has certain rights in this invention.

All patent applications, published patent applications, issued and granted patents, texts, and literature references cited in this specification are hereby incorporated herein by reference in their entirety.

### BACKGROUND OF THE INVENTION

The budding yeast Saccharomyces cerevisiae is an excellent system for the study of many problems of eukaryotic biology. Despite the diverse nature of the questions asked, almost all of these studies use molecular tools and often require the transfer of exogenous DNA into the yeast cell. Several techniques for high-efficiency transformation of genetic material into yeast have been developed and used successfully over the years. These include spheroplast transformation, transformation by electroporation and lithium acetate transformation. One of the major drawbacks of these methods is the many and often time consuming steps that are necessary to prepare the host strain, introduce the exogenous DNA and identify successful transformants. This becomes an important issue when the transformation of extrachromosomal DNA such as a plasmid into a large number of host strains is desired. Modifications that scale down the lithium acetate method have been successfully used for large-scale transformation of the same plasmid into a set of strains. However, transformation efficiency is compromised and this method may not be useful for all purposes.

The problem becomes even more acute when multiple distinct DNAs need to be transferred from one strain to another since this requires the extra steps of DNA isolation from yeast and plasmid rescue in bacteria. In the age of genome-scale manipulations of yeast, an easier and less time consuming method of plasmid introduction or transfer is desirable. Previous work described a method of kar-mediated transfer that allows the quick and efficient transfer of plasmids from one yeast strain to another or to a number of different strains. The method is based on yeast mating and uses a particular mutation in the karyogamy pathway, kar1(Conde and Fink, 1976). kar1 mutant strains initiate mating and proceed through conjugation without nuclear fusion, and therefore haploid progeny can be recovered. During a kar mating heterokaryons with mixed cytoplasm are formed and extrachromosomal nuclear DNA such as plasmids or YACs can be transferred between the two parents (Dutcher 1981). With the proper selection of nuclear genetic markers, which allow for the selection of one strain (recipient) and the counterselection of the other (donor), plasmid transfer can be made directional.

Most laboratory yeast strains exist as MATa or MATα haploids. A mating between MATa and MATα yeast produces diploids. This process begins when haploid cells of the opposite mating type adhere to one another after the mutual exchange of pheromones. This is followed by the orderly removal of cell walls and plasma membrane to complete cell fusion (Marsh and Rose 1997). One characteristic feature of all fungi including yeast is that during the process of conjugation, the nuclear envelope remains intact and the resulting diploid nucleus is the product of the direct fusion of the two parental nuclei.

A number of mutations that block the karyogamy pathway have been identified and studied. One such mutation is kar1-1, which prevents nuclear fusion in approximately 90-95% of matings (Rose and Fink, 1987). KAR1 is essential for mitotic growth because of its role in the initial stage of spindle pole body duplication. Kar1 has three functional domains that separate its mitotic function from its role during nuclear fusion (Vallen et al., 1992). An amino-terminal domain is important for the nuclear fusion function and a deletion of this protein domain gives an allele, kar1Δ15, that is viable and defective only in nuclear fusion. kar1Δ15 mutation is unilateral in that its defect is observed even in a mating to a wild type (WT) strain.

Matings in which either parent is kar1Δ15 mutant are unproductive since they generate diploid nuclei at a very low frequency. The majority of the products from such matings are cytoductants and, in practice, they can be selected when the nuclei and the cytoplasm of each parent are marked genetically. Another characteristic of kar mating, of particular interest to the technique described here, is the occasional transfer of genetic material from one nucleus to the other. This property of kar mating has been used for the directional transfer of yeast artificial chromosomes from one strain to another host of interest. Although kar1 matings can efficiently move plasmids between strains, there are two problems for use especially when extrachromosomal material such as plasmids are to be transferred into multiple recipient strains. One of the major problems of the previously described method of kar-mediated transfer is that transfer of the desired genetic material is often accompanied by an undesirable co-transfer of a donor chromosome as well. Chromosome co-transfer was shown to be as high as 4% per chromosome during YAC chromoduction with as many as 20% of the total chromoductants receiving an extra chromosome (Spencer et al. 1994). We have also shown that this is the case during plasmid transfer.

An additional problem is that the previously used methods require a particular marker configuration in both the donor and recipient strains to permit the selection of the recipient nucleus. As outlined in FIG. 1, the standard experiment requires two recessive drug resistance markers (cyh2^{R} and can1^{R}) in the recipient strain. This becomes an obvious problem when the method of kar-mediated transfer is used for transferring genetic material into multiple strains. For example, the current gene disruption library strains are CYH^{S} and CAN^{S}. Thus, for each library strain, spontaneous cyh2^{R} and can1^{R} alleles need to be isolated by selection on drug medium. Although this is possible in principle, it requires multiple manipulations of the whole library and it is impractical since each strain would have to be confirmed before it could be used.

### SUMMARY OF THE INVENTION

The present invention improves the technique of directional transfer of extrachromosomal genetic material between yeast strains. In one embodiment, the present invention solves the problem of undesirable chromosome co-transfer by providing a universal donor strain in which all sixteen yeast chromosomes carry a counterselectable cassette, which comprises a counterselectable marker and a regulatable promoter which can conditionally destabilize the centromere when transcription is active through the centromere. The presence of the counterselectable cassette on each chromosome allows for the counter selection against each donor chromosome. The invention provides a method for making a universal donor strain. The universal donor strain further improves previous methods of kar-mediated transfer by alleviating the need for the presence of recessive drug markers in the recipient strain. Kar-mediated transfer using the universal donor of the invention only requires that the recipient strain lacks the counterselectable marker of the donor chromosome, is capable of supporting transcription through the centromere and has another marker available for the selection of the transferred genetic material. The requirements of the recipient strain are easily met by many laboratory yeast strains.

The invention provides for a yeast cell comprising chromosomes I through XVI, wherein at least two chromosomes comprise a nucleic acid sequence encoding a counterselectable marker linked to a centromere. In another embodiment, the invention provides for a yeast cell comprising chromosomes I through XVI, wherein each chromosome comprises a nucleic acid sequence encoding a counterselectable marker linked to a centromere. The yeast cell may be a donor cell. It can have every chromosome marked with a counterselectable marker, such that if a chromosome were to be transferred to a recipient cell, the recipient cell can be selected against (counterselected). In one embodiment, the nucleic acid sequence further comprises a promoter capable of promoting transcription into the centromere of the chromosome, wherein the promoter is inducible or repressible. In a further embodiment, the counterselectable marker comprises a URA3 gene and the promoter comprises a GAL1 promoter. In one aspect of the invention, the yeast cell can be of the genus Saccharomyces.

In another aspect of the invention, the yeast cell comprises an extrachromosomal nucleic acid comprising a dominant selectable marker, and wherein the yeast genome lacks the selectable marker. In another embodiment, lack of the selectable marker in the cell creates a metabolic deficiency or drug sensitivity. In one embodiment, the selectable marker comprises LEU2, LYS5, TRP1, HIS3 prototrophic gene, the heterologous drug resistance marker KanMX or any combination thereof.

In one embodiment, the yeast cell further comprises a mutation in the karyogamy pathway which permits yeast mating while preventing nuclear fusion.

The invention also provides for a method for transferring extrachromosomal genetic material into a recipient yeast cell, the method comprising: a) mating a recipient yeast cell to the donor yeast cell of the invention, wherein at least one of the cells comprises a mutation in the karyogamy pathway, b) selecting against the donor cell via the counterselectable marker, and c) selecting for recipient cells which have acquired the extrachromosomal genetic material. The invention also provides for a method for transferring extrachromosomal genetic material into a recipient yeast cell, the method comprising: a) mating a recipient cell to a donor yeast cell of the invention, wherein the donor cell comprises a mutation in the karyogamy pathway, b) selecting against the donor cell, and c) selecting for recipient cells which have acquired the extrachromosomal genetic material. In one embodiment, the extrachromosomal genetic material comprises comprises a plasmid, an episomal nucleic acid, a mitochondrial nucleic acid or a virus. In one aspect of the invention, the methods described herein can be carried out using automation, wherein the steps are automated.

The invention also provides for a method for transferring extrachromosomal material into a recipient yeast cell comprising the steps of: a) mating a recipient cell to the donor yeast cell of the invention, wherein at least one of the cells comprises a mutation in the karyogamy pathway, b) selecting against the donor cell, and c) screening for recipient cells which have acquired the extrachromosomal material.

The invention also provides for a method for transferring extrachromosomal material into a recipient yeast cell, the method comprising: a) mating a recipient cell to a donor yeast cell of the invention, wherein the donor cell comprises a mutation in the karyogamy pathway, b) selecting against the donor cell, and c) screening for recipient cells which have acquired the extrachromosomal material. In one embodiment, the extrachromosomal material comprises cytoplasm, a protein, a prion, an organelle or any combination thereof.

The invention also provides for a method for making the yeast cell of invention, the method comprising: a) providing a diploid yeast cell that has one copy of chromosomes I through XVI marked with a counterselectable nucleic acid linked to the centromere, wherein the counterselectable nucleic acid comprises URA3, b) sporulating the diploid, c) plating the sporulated diploid to generate an original colony of adjacent haploid cells derived from the spores in an single ascus, d) identifying an original colony which comprises spores that are uracil auxotrophs resistant to 5-FOA via growth on 5-FOA containing medium, e) isolating uracil prototrophs spores that are adjacent to the uracil auxotrophs spores, and f) performing a diagnostic PCR to confirm that all sixteen chromosomes in the uracil prototroph have the nucleic acid sequence which comprises URA3.

In another embodiment, the invention provides for a method for identifying a nucleic acid sequence that exhibits synthetic dosage lethality in a yeast cell, the method comprising: a) mating a recipient yeast cell to a donor yeast cell of the invention, wherein at least one of the donor and recipient cells comprises a mutation in the karyogamy pathway, b) selecting against the donor cell, c) selecting for recipient cells which have acquired genetic material transferred from the donor cell, and d) identifying recipient cells which fail to grow due to failure to acquire the genetic material transferred from the donor cell. In one aspect, the invention provides that the donor cell expresses a human homologue of a gene associated with a proliferative disease. In another embodiment, the proliferative disease comprises cancer. In another embodiment, the recipient yeast cell comprises a cell from a yeast gene knockout library. In another embodiment, the invention provides for a method for transferring a yeast chromosome of interest from a donor yeast cell into a recipient yeast cell, the method comprising: a) mating a recipient yeast cell to the donor yeast cell of the invention, wherein at least one of the cells comprises a mutation in the karyogamy pathway, b) selecting against the donor cell via the counterselectable marker, and c) selecting for recipient cells which have acquired the yeast chromosome of interest, wherein the yeast chromosome of interest does not contain the counterselectable marker.

### BRIEF DESCRIPTION OF THE DRAWINGS

FIG. 1 shows the method of kar-mediated transfer of genetic material as previously practiced.

FIG. 2A shows the improved method of kar-mediated transfer using the universal donor strain of the invention.

FIG. 2B shows one embodiment of the CEN::URA3 GAL cassette inserted into each Saccharomyces chromosome. Thick tapered black lines represents the chromosome sequence and the dashed lines indicate the insertion of the cassette into the chromosome directly adjacent to the centromere (represented as a dark circle). The white box represents the URA3 gene from Kluyveromyces lactis that fully complements a S. cerevisiae ura3 mutation. The arrow indicates transcription from the GAL1 promoter through the centromere.

FIG. 3B shows in schematic the construction of a sixteen-tuple URA3 strain using a method of the invention. Four rounds of crosses are necessary to construct this strain. Panel A shows one of the eight crosses necessary in the first round of the construction. The URA3+ centromere linked marker is depicted as a triangle and plus sign. The diploids are heterozygous for these two insertions and only ditype tetrads arise after meiosis and sporulation. One half of the tetrads have the desired 2:2 configuration as shown on SD-uracil and 5FOA plates. One of the four crosses from Round 2 is shown in panel B. Since the four URA3+ markers are centromere-linked, eight ditype tetrads are possible. The circle indicates the desired 2:2 configuration which occurs in two of the 12 tetrads shown on the right. C. Round 3 and 4 crosses are not diagrammed, but probabilities for recovering the non-parental configuration of markers that result in 2:2 segregation of uracil prototrophy are shown for each round of crosses. See text for details of isolation of the correct segregants

FIGS. 4A-4C show the results of PCR analysis which demonstrates the construction of the sixteen-tuple URA3 strain. A. A generic centromeric region for a yeast chromosome is diagrammed as a thick line with the centromere indicated by a dark circle. Vertical dashed lines indicate the extent of the homology region used to integrate the URA3 cassettes. Diagnostic PCR reactions are indicated by arrows flanking dashed lines, and letters above those lines refer to the ethidium stained gels showing PCR products in B or C. PCRs in B used a common primer within the URA3 gene and 16 different chromosome-specific primers for the individual reactions and could give no product if the URA3 integration were not present. The C PCR reactions could result in short products for an unmarked (wild-type) centromere. Long products were possible by amplification through a URA3 cassette, but were rare due to the short extension times used for the reactions. B. Ethidium stained gel showing 16 chromosome-specific PCRs using the “B” primer set on DNA from two isolates from the tetrad screen (W4730-121 & W4730-257). All 16 markers are amplified in these reactions. Control reactions were performed on strains W3199 and W3200 which contained marked chromosomes 1-8 and 9-16 respectively. The PCR reactions in each lane are specific to the chromosome numbered on that lane (e.g., strain 3199 does not contain URA3 markers on chromosomes 9-12). C. Control PCR reactions using primer set C on wild-type and W4730-257 DNA. All 16 centromere PCRs result in short products for a wild-type strain. In most cases, strain W4730-257 results in no product due to a short extension reaction. A product is evident in the reaction using chromosome 14 primers but matches the size for a URA3-marked chromosome. The same reactions using strain W4730-121 resulted in no PCR products indicating that neither 16-tuple strain contained an unmarked chromosome

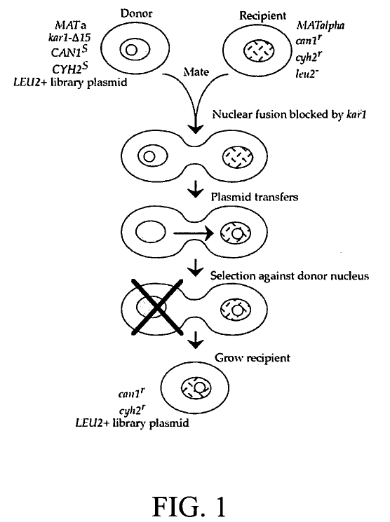

TABLE 1

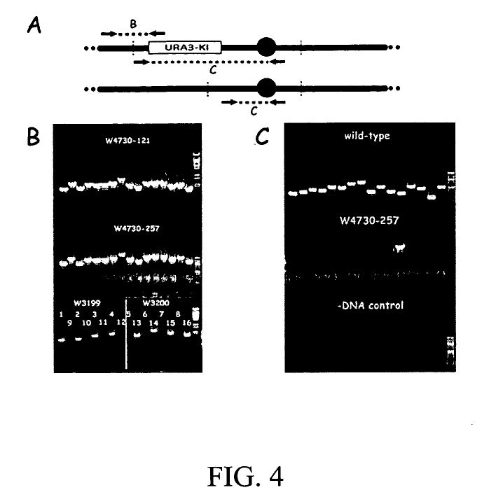

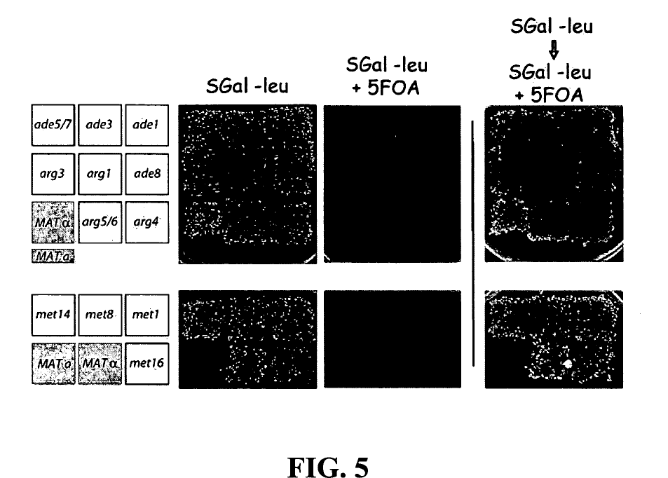

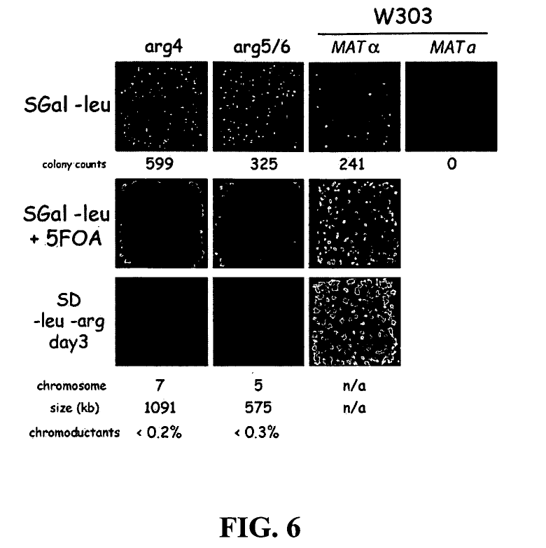

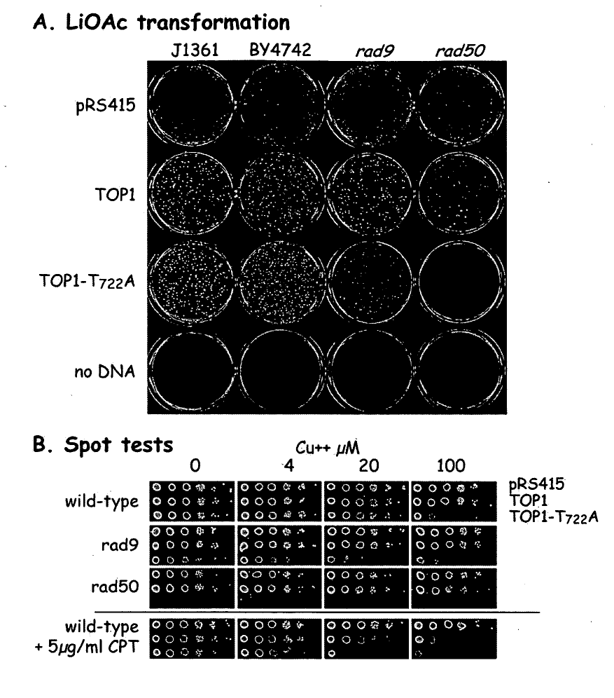

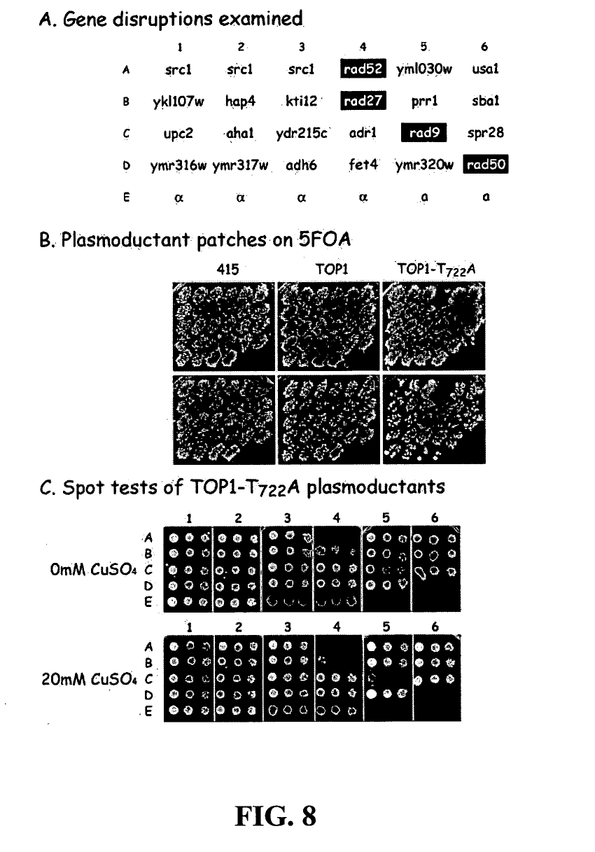

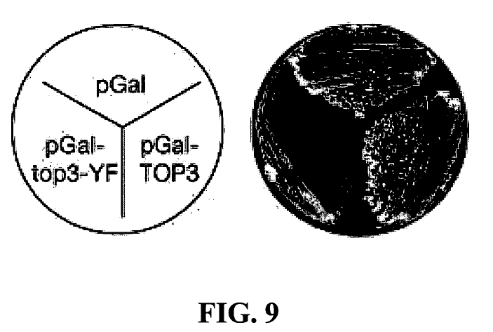

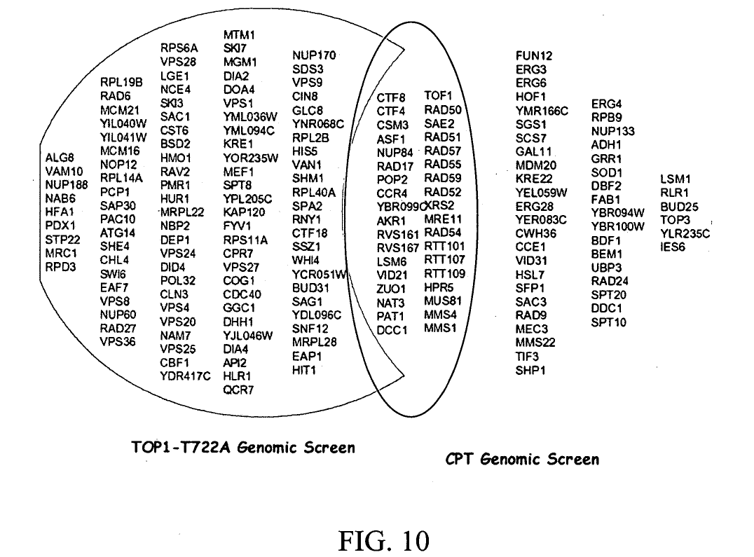

PCR primers used in FIG. 4.

| | |
|---|---|
| Primer | 5′ to 3′ |

| | | |
|---|---|---|
| name | Nucleotide Sequence | Purpose |

| | | |
|---|---|---|
| CEN1F-out | GATACGTCCAGTCACACTTC | Chromosome 1-specific primer for PCR reaction “B” in FIG. 4. |
| CEN2F-out | GTTCCTGCACTGTGTAAAGTG | Chromosome 2-specific primer for PCR reaction “B” in FIG. 4. |
| CEN3F-out | GCAATCGACTCCAAATCAAACTC | Chromosome 3-specific primer for PCR reaction “B” in FIG. 4. |
| CEN4F-out | GTGACAAGCACCCAGATCTG | Chromosome 4-specific primer for PCR reaction “B” in FIG. 4. |
| CEN5F-out | CTGATATGGCAGCACAGAAG | Chromosome 5-specific primer for PCR reaction “B” in FIG. 4. |
| CEN6F-out | GAGCAGCTAATACGAAGGATC | Chromosome 6-specific primer for PCR reaction “B” in FIG. 4. |
| CEN7F-out | GCACTAGTAACCCTCAATCTC | Chromosome 7-specific primer for PCR reaction “B” in FIG. 4. |
| CEN8F-out | CCTTCAATTGCATCATAGGTAC | Chromosome 8-specific primer for PCR reaction “B” in FIG. 4. |
| CEN9F-out | CTGTTCACAGCGCATAAGTTC | Chromosome 9-specific primer for PCR reaction “B” in FIG. 4. |
| CEN10-out | GAATGGAATTCAAGTGGACATC | Chromosome 10-specific primer for PCR reaction “B” in FIG. 4. |
| CEN11F-out | CTCTGTCAACTCTGTATGGAG | Chromosome 11-specific primer for PCR reaction “B” in FIG. 4. |
| CEN12F-out | CTGTTCTATTTCCCTAATGCTC | Chromosome 12-specific primer for PCR reaction “B” in FIG. 4. |
| CEN13F-out | CAGTCGTTACAGAGGAACAG | Chromosome 13-specific primer for PCR reaction “B” in FIG. 4. |
| CEN14F-out | GCATTCGTTGCTGCATCGTTG | Chromosome 14-specific primer for PCR reaction “B” in FIG. 4. |
| CEN15F-out | GGAAGACCGATTGCTTTAGAC | Chromosome 15-specific primer for PCR reaction “B” in FIG. 4. |
| CEN16F-out | GAGAATGCATCGTCGTGTTC | Chromosome 16-specific primer for PCR reaction “B” in FIG. 4. |
| GAL-R2 | ATTGACGGGAGTGTATTGACGC | Common “reverse” PCR primer to verify centromere-proximal |
| | | insertions |
| CEN1F | CATAAGTGTGCCTTAGTATGC | Chromosome 1-specific forward primer for PCR reaction “C” in |
| | | FIG. 4. |
| CEN2F | CATTTCCCAAGAGGATCAATC | Chromosome 2-specific forward primer for PCR reaction “C” in |
| | | FIG. 4. |
| CEN3F | CCAACAGATATAGGCTGTGTC | Chromosome 3-specific forward primer for PCR reaction “C” in |
| | | FIG. 4. |
| CEN4F | CTATGCTGTCTCACCATAGAG | Chromosome 4-specific forward primer for PCR reaction “C” in |
| | | FIG. 4. |
| CEN5F | CCATCAAGCCCATTCAATGC | Chromosome 5-specific forward primer for PCR reaction “C” in |
| | | FIG. 4. |
| CEN6F | CGATGGAAGAGGTAAAGTAGTC | Chromosome 6-specific forward primer for PCR reaction “C” in |
| | | FIG. 4. |
| CEN7F | GTACTGGTGAAAGAATGTCTG | Chromosome 7-specific forward primer for PCR reaction “C” in |
| | | FIG. 4. |
| CEN8F | CTCCAACAATTACACATCCA | Chromosome 8-specific forward primer for PCR reaction “C” in |
| | | FIG. 4. |
| N-CEN9F | TTCTCTCCGCCAGGAAACTGA | Chromosome 9-specific forward primer for PCR reaction “C” in |
| | | FIG. 4. |
| CEN10F | CGACAAAGTATCTCAGAAGGG | Chromosome 10-specific forward primer for PCR reaction “C” in |
| | | FIG. 4. |
| CEN11F | GAAGTGCAGAGTTATCTGCTAC | Chromosome 11-specific forward primer for PCR reaction “C” in |
| | | FIG. 4. |
| CEN12F | GAAGGTTCATATTCTGTGAACGC | Chromosome 12-specific forward primer for PCR reaction “C” in |
| | | FIG. 4. |
| CEN13F | CTTGTAGCTGTTGAGCTGTAC | Chromosome 13-specific forward primer for PCR reaction “C” in |
| | | FIG. 4. |
| CEN14F | GAAACTGATGGACTCCGTAG | Chromosome 14-specific forward primer for PCR reaction “C” in |
| CEN15F | GTACATCCGGATTTTCAGAAG | Chromosome 15-specific forward primer for PCR reaction “C” in |
| CEN16F | CTGATCCAGAAAAGGCAAGAG | Chromosome 16-specific forward primer for PCR reaction “C” in |
| | | FIG. 4. |
| CEN1R | CCGCCTAGTGCTTAAGAGTTC | Chromosome 1-specific reverse primer for PCR reaction “C” in |
| | | FIG. 4. |
| CEN2R | GATCTGTATATCGTCGTCACC | Chromosome 2-specific reverse primer for PCR reaction “C” in |
| | | FIG. 4. |
| CEN3R | CTTCCACCAGTAAACGTTTC | Chromosome 3-specific reverse primer for PCR reaction “C” in |
| | | FIG. 4. |
| CEN4R | CCTCTTTATATGATCTGCCG | Chromosome 4-specific reverse primer for PCR reaction “C” in |
| | | FIG. 4. |
| CEN5R | GTTGAAACGCCAACAGTGGC | Chromosome 5-specific reverse primer for PCR reaction “C” in |
| | | FIG. 4. |
| CEN6R | CATCGTAAACGTGTGTAGAGC | Chromosome 6-specific reverse primer for PCR reaction “C” in |
| | | FIG. 4. |
| CEN7R | CGATCTATAGGGACTATCGG | Chromosome 7-specific reverse primer for PCR reaction “C” in |
| | | FIG. 4. |
| CEN8R | CTAAGTTCGGAACACAAAACCC | Chromosome 8-specific reverse primer for PCR reaction “C” in |
| | | FIG. 4. |
| N-CEN9R | TCTCGCTTGTCTATCCAAACCTTC | Chromosome 9-specific reverse primer for PCR reaction “C” in |
| | | FIG. 4. |
| CEN10R | CTCGTTCTCAACAACTCTAC | Chromosome 10-specific reverse primer for PCR reaction “C” in |
| | | FIG. 4. |
| CEN11R | CCTAATACCTCAATGGTCCAATAC | Chromosome 11-specific reverse primer for PCR reaction “C” in |
| | | FIG. 4. |
| CEN12R | GCAGATAGACCTTCTACATGG | Chromosome 12-specific reverse primer for PCR reaction “C” in |
| | | FIG. 4. |
| CEN13R | CTAAGGTAGCCAGAACTTCTC | Chromosome 13-specific reverse primer for PCR reaction “C” in |
| | | FIG. 4. |
| CEN14R | GCTGCACGTGACTAACTAG | Chromosome 14-specific reverse primer for PCR reaction “C” in |
| | | FIG. 4. |
| CEN15R | CATCATAAGAGCATAAGCGACAG | Chromosome 15-specific reverse primer for PCR reaction “C” in |
| | | FIG. 4. |
| CEN16R | GCCGATTTCGCTTTAGAACC | Chromosome 16-specific reverse primer for PCR reaction “C” in |
| | | FIG. 4. |

FIG. 5 shows plasmoduction of a LEU2-marked plasmid into 14 strains. The universal MATa donor strain (J1361) was transformed with a yeast shuttle vector marked with the wild-type LEU2 gene using standard transformation procedures. Transformants were grown in liquid SD-leu media overnight, spread on SD-leu agar plates and incubated overnight to grow a lawn. MATa and MATalpha W303 strains (Grey boxes) and 12 MATalpha strains from the yeast gene disruption library were patched onto YPD plates for overnight growth as indicated in the cartoon on the left. Matings were performed by replica transfer of the donor lawn and the patches to a YPD plate for 6 hours at 30° C. The matings were replica plated to synthetic galactose media lacking leucine (Sgal-leu) or Sgal-leu that also contained 5FOA (Sgal-leu+5FOA). The LEU+ colonies growing in the presence of galactose indicate mating-dependent transfer of the LEU2 plasmid to the recipient strain (note that no colonies are growing from the MATa patches). No colonies grow on the Sgal-leu+5FOA plates suggesting that immediate 5FOA selection is too stringent for the plasmoduction procedure (see text). In contrast, serial selection on Sgal-leu then on Sgal-leu+5FOA results in many plasmoductant colonies.

FIG. 6 shows an assay to detect spurious allele transfer using the universal donor strain. A LEU2 vector was transferred from the universal donor into the indicated strains by plasmoduction as described in FIG. 5. arg4 and arg5/6 strains are MATalpha auxotrophs from the yeast gene disruption library. The W303 strains are arginine prototrophs of the indicated mating type. Sgal-leu indicates the first round of selection for plasmoductants. Numbers below each panel are the colonies counted from each patch. Sgal-leu+5FOA is the second round of selection and all colonies grow on this medium. The third row shows replica plating from the 5FOA plates to synthetic media lacking leucine and arginine. The W303 recipient is ARG+ so the colonies grow on this medium. The library strains used in this experiment only grow if a wild-type allele was able to co-transfer from the W303 donor along with the plasmid.

FIG. 7 shows the sensitivity of checkpoint and repair-deficient strains to expression of TOP1-T₇₂₂A. A. The TOP1-T₇₂₂A mutation alters Top1 enzyme catalysis—mimicking the effect of the chemotherapy drug camptothecin—and causes DNA breaks. The mutant and wild-type TOP1 were cloned under the control of the copper-inducible CUP1 promoter. These plasmids were transformed into the MATa universal donor strain (J1361), the MATalpha deletion library wild-type strain (BY4742) and rad9 and rad50 deletion strains from the library. Transformation reactions were plated on SD-leu media and incubated for 3 days at 30° C. to allow colony growth. The vector control (pRS415) and TOP1 plasmids transform efficiently into each of the strains. The TOP1-T₇₂₂A mutant transforms efficiently into all but the rad50 deletion mutant. Although no copper is added to the SD-leu plates, leaky expression of TOP1-T₇₂₂A from the CUP1 promoter is sufficient to kill any rad50 cells that were transformed. B. Transformants from A were grown overnight in liquid SD-leu media, equalized by OD, serially diluted and spotted onto SD-leu plates containing the indicated concentrations of CuSO₄. Each panel is a group of three strains containing the vector, TOP1 or TOP1-T₇₂₂A plasmids as indicated on the right. The vector and TOP1 containing strains grow well at all concentrations of copper tested. Wild-type yeast containing the TOP1-T₇₂₂A plasmid shows decreased viability at 100 μM copper while rad9 strains start showing decreased viability at 20 μM copper, indicating a greater sensitivity to TOP1-T₇₂₂A expression in this checkpoint deficient strain. Spot tests shown at the bottom contain 5 μg/ml camptothecin. At this dose, wild-type strains are only sensitive to the drug when Top1 is overexpressed by 100 μM copper. There is a roughly additive effect of the TOP1-T₇₂₂A mutant in conjunction with camptothecin in wild-type cells as the bottom set of spots shows sensitivity at 20 μM copper.

FIG. 8 shows a pilot plasmoduction screen for gene disruptions sensitive to TOP1-T₇₂₂A expression. A. Gene disruption strains from the MATalpha library were patched onto a YPD plate for plasmoduction. 4 sets (A-D) of 6 strains (1-6) from adjacent library grid positions were tested. Each of these sets contains a gene disruption highlighted in black that is known to be camptothecin sensitive. The bottom row (E) contains MATalpha and MATa wild-type strains as plasmoduction controls. B. The universal donor strain containing the vector control (415), TOP1 or TOP1-T₇₂₂A was mated to the grid of strains in duplicate and plasmoductants were selected as described in FIG. 5. Colonies after the final replica and growth on selection plates (5FOA) are shown. The vector control and TOP1 plasmids transfer into every strain except the MATa controls (lower right two patches). The TOP1-T₇₂₂A plasmid does not transfer into the rad52 (A4) or rad50 (D6) strains. C. Colonies were picked from the plasmoductant plates, grown overnight in liquid SD-leu media, serially diluted and spotted onto SD-leu plates with 0 or 20 μM copper. Only the TOP1-T₇₂₂A spot tests are shown. The 0 μM results mirror the patches in B where rad50 and rad52 spots are missing. In the 20 μM spots the rad9 and rad27 strains show about 100-fold less growth due to higher TOP1-T₇₂₂A expression.

FIG. 9 shows that expression of the top3-Y₃₆₅F catalytic mutant confers slow growth. The catalytic mutant top3-Y₃₆₅F was constructed by PCR using primers containing the site specific mutation. The wild-type and Y₃₆₅F alleles were cloned in front of the pGAL1 promoter and transformed into a diploid W303 strain. Transformants were picked and restreaked to plates containing galactose. Expression of the top3-Y₃₆₅F mutant produces slow growth similar to a top3 null mutant while expression of the wild-type allele has no effect on growth (compare with vector control).

FIG. 10 shows the overlap and the difference between the set of mutants identified from the SDL screen with Top1-T722A of Example 1 and the set of camptothecin sensitive mutants identified in the CPT genomic screen (Parsons et al., 2004).

### DETAILED DESCRIPTION OF THE INVENTION

### Terms

As used herein the term “karyogamy” refers to the process of nuclear congression and fusion during conjugation

As used herein the term “kar mutants” refers to mutants defective in the karyogamy pathway. In one embodiment kar mutations lead to cytoplasmic fusion but no nuclear fusion.

As used herein the term “kar mating” refers to a mating in which one of the parents carries a particular kar mutation. In one embodiment a mutation in this context is kar1Δ15.

As used herein the term “recipient strain” refers to the parent strain (nucleus) that is selected as a host for the transferred material (plasmid). The recipient can be haploid.

As used herein the term “donor strain” refers to the strain that carries the material (plasmid) that is transferred to the recipient via a kar mating. The donor can be a haploid strain.

As used herein the term “universal donor strain” refers to a donor strain which comprises yeast chromosomes I through XVI marked with a counterselectable cassette.

As used herein the term “cytoductants” refers to the haploid progeny from a kar mating with the nucleus of the recipient and mixed cytoplasm from both parents.

As used herein the term “chromoductants” refers to the haploid progeny from a kar mating with the nucleus of the recipient and a chromosome(s) transferred from the donor.

As used herein the term “plasmoductants” refers to the haploid progeny from a kar mating with the nucleus of the recipient and a plasmid from the donor.

As used herein the term “YACductants” refers to the haploid progeny from a kar mating with the nucleus of the recipient and a YAC from the donor.

As used herein the term “selectable marker” refers to a gene and its product which allows for the positive selection of cells that have the gene product.

As used herein the term “counterselectable marker” refers to a gene and its product which allows for the selection against those cells that have the gene product. This terms includes functional variants of the wild type copy of the gene. Certain gene products can be both selectable and counterselectable markers, non-limiting examples are URA3, TRP1, LYS5 markers.

As used herein the term “complementing copy” means a copy of the gene that functionally complements a mutation.

The invention is an improvement on a method that was originally used for YAC transfer using kar1 mutant strains (Spencer et al. 1994). kar1Δ15 strains mate but have a defect in nuclear fusion. Nevertheless, in the absence of nuclear fusion, some DNA can transfer from one nucleus to the other in the zygote and appropriate selections can be used to recover a haploid strain containing the recipient nucleus and the YAC of interest. This idea was adopted and expanded to move plasmids between strains. It is efficient because matings can be simply performed by replica plating, which is well suited to moving a test plasmid into a library of strains (1 into many) or a library of plasmids into a new assay strain (many into 1) (Georgieva and Rothstein 2002). The new donor strain of the invention greatly improves on the YAC transfer method by simplifying the genetic requirements of the recipient strain. Importantly, the universal donor strain used in the method limits spurious chromosome transfer from the donor to the recipient—a known problem with YAC transfers (Dutcher 1981; Hugerat et al. 1994).

The karyogamy mutation used in the method of kar transfer is the kar1Δ15 allele. However, any other mutation that allows plasma membrane fusion, initiates nuclear congression but prevents nuclear fusion is also contemplated for use in the method of the present invention. Since the kar1Δ15 is unilateral in that its effect is observed in a mating to a wild type stain, the kar1Δ15 mutation can be present in either the donor or the recipient strain. In one embodiment, the kar1Δ15 mutation is introduced in the universal donor strain.

All mating and selections are performed by replica transfer of the donor and recipient strains, so it is possible to perform many matings at once, for instance with strains arrayed in 96-well format on an agar plate. Matings and selection can also be performed in liquid media. Standard yeast media and methods of yeast manipulation are described in Sherman et al., 1986.

The most important prerequisite for a directional transfer of extrachromosomal genetic material in a kar-mediated transfer is the proper design of the strains. The goal is to select haploid progeny that contain the desired parental nucleus and that have acquired the extrachromosomal genetic material of interest. One consideration is that the recipient is auxotrophic for the marker selecting the plasmid. The other consideration is that the parental genotypes allow selection for the recipient and against the donor nucleus.

The present invention solves problems of previously used methods of kar-mediated transfer. The two major problems of the method as previously used are the spurious transfer of donor chromosomes and the frequent necessity for extensive genetic manipulation of the recipient strain to allow for the directional transfer of genetic material. The invention as described below, overcomes both of these problems by constructing a new universal donor strain. A universal donor strain as contemplated by the invention comprises yeast chromosomes that comprise a counterselectable cassette that is genetically linked to the centromere of the chromosome. In one embodiment all sixteen yeast chromosomes have the counterselectable cassette.

### Universal Donor Strain

An important consideration in preventing spurious transfer of chromosomes from the donor is the ability to select against the donor chromosomes. In one aspect the invention provides a donor strain in which chromosomes are marked with a counterselectable cassette (a nucleic acid sequence encoding a counterselectable marker) which permits for the selection against donor cells. The counterselectable cassette is genetically linked to the centromere of the chromosome. Furthermore, the donor strain is auxotroph for the selectable marker borne on the extrachromosomal genetic material.

In one embodiment, the counterselectable cassette comprises a nucleic acid sequence which encodes a counterselectable marker. In another embodiment the counterselectable cassette comprises a counterselectable marker and a conditional centromere. The conditional centromere allows the regulated destabilization of the corresponding chromosome. In one embodiment the conditional centromere has a regulatable promoter that can drive transcription into the centromere. Active transcription through the centromere is thought to destabilize the centromere thus eventually resulting in chromosomal loss. The regulatable promoter can be an inducible promoter and a number of inducible promoters, for example but not limited to GAL1/10, O, CUP1, etc., are contemplated for use in the invention. In one embodiment the inducible promoter is GAL1 which expression is induced in a GAL+ stain in the presence of galactose. In another embodiment the regulatable promoter can be a repressible promoter. The repressible promoter can be the tetracycline repressible promoter which is turned off in the presence of tetracycline and turned on when tetracycline is removed from the growth media. The presence of a counterselectable marker allows for the selection against the cells that carry this marker. A number of counterselectable markers, for example but not limited to URA3, LYS5, TRP1, etc., can be used in the invention. In one embodiment the counterselectable marker is the URA3 gene, which can be the Kluyveromyces lactis URA3 gene which complements the uracil auxotrophy of Saccharomyces strains. URA3+ strains are unable to grow in the presence of the pyrimidine analogue 5-fluororotic acid (5-FOA). Therefore, the presence of the URA3 gene product allows counter-selection of yeast cells grown on medium containing 5-FOA. The compound alpha amino adipic acid can be toxic to LYS5+ strains. The drug 5-fluoroanthranilic acid (5-FAA) is toxic to TRP1+ strains. An important aspect of the invention is that the counterselectable marker of the centromeric cassette has to be different from the selectable marker used to mark the extrachromosomal genetic material.

In one embodiment, the nucleic acid sequence that encodes the counterselectable marker and the regulatable promoter can be genetically linked to the centromere and both can be on the same chromosomal arm. In another embodiment, the nucleic acid sequence that encodes the counterselectable marker and the regulatable promoter can be genetically linked to the centromere and can be on each side of the centromere on each chromosomal arm. The counterselectable cassette can be inserted at any position near the centromere as long as the counterselectable marker is linked to the centromere and the regulatable promoter can drive transcription into the centromere.

In one embodiment the nucleic acid sequence that encodes the counterselectable marker and the regulatable promoter is inserted at position within 50 base pairs on either side of the centromere. In another embodiment the nucleic acid sequence that encodes the counterselectable marker and the regulatable promoter is inserted at position which is between 50 to 100 base pairs on either side of the centromere. In another embodiment the nucleic acid sequence that encodes the counterselectable marker and the regulatable promoter is inserted at position which is between 101 to 200 base pairs away on either side of the centromere. In another embodiment the nucleic acid sequence that encodes the counterselectable marker and the regulatable promoter is inserted at position which is between 201 to 300 base pairs away on either side of the centromere. In another embodiment the nucleic acid sequence that encodes the counterselectable marker and the regulatable promoter is inserted at position which is between 301 to 400 base pairs away on either side of the centromere. In another embodiment the nucleic acid sequence that encodes the counterselectable marker and the regulatable promoter is inserted at position which is between 401 to 500 base pairs away on either side of the centromere. In another embodiment the nucleic acid sequence that encodes the counterselectable marker and the regulatable promoter is inserted at position which is between 501 to 600 base pairs away on either side of the centromere. In another embodiment the nucleic acid sequence that encodes the counterselectable marker and the regulatable promoter is inserted at position which is between 601 to 700 base pairs away on either side of the centromere. In another embodiment the nucleic acid sequence that encodes the counterselectable marker and the regulatable promoter is inserted at position which is between 701 to 800 base pairs away on either side of the centromere. In another embodiment the nucleic acid sequence that encodes the counterselectable marker and the regulatable promoter is inserted at position which is between 801 to 900 base pairs away on either side of the centromere. In another embodiment the nucleic acid sequence that encodes the counterselectable marker and the regulatable promoter is inserted at position which is between 901 to 1000 base pairs away on either side of the centromere. In another embodiment the nucleic acid sequence that encodes the counterselectable marker and the regulatable promoter is inserted at position which is between 1001 to 1500 base pairs away on either side of the centromere.

In one embodiment the nucleic acid sequence that encodes the counterselectable marker and the regulatable promoter is inserted at position 151438 of chromosome I, at position 238095 of chromosome II, at position 114317 of chromosome III, at position 449664 of chromosome IV, at position 151909 of chromosome V, at position 148447 of chromosome VI, at position 497118 of chromosome VII, at position 105736 of chromosome VIII, at position 355545 of chromosome IX, at position 435911 of chromosome X, at position 439693 of chromosome XI, at position 151015 of chromosome XII, at position 267929 of chromosome XIII, at position 629030 of chromosome XIV, at position 326788 of chromosome XV, and at position 555857 of chromosome XVI.

The nucleic acid sequence of the counterselectable cassette comprising the URA3 marker and GAL1 promoter is the following:

LOCUS INSERT_SEQ 2205 BP DS-DNA SYN 24-AUG-2005
DEFINITION -
ACCESSION -
KEYWORDS -
SOURCE -
FEATURES Location/Qualifiers
CDS 372 . . . 1175
/note = “K. lactis URA3”
promoter 1514 . . . 2185
/note = “GAL1-10 promoter”
promoter 2144 . . . 2151
/note = “GAL1 TATA box”
BASE COUNT 684 A 428 C 450 G 643 T 0 OTHER
ORIGIN -

| | | | | | | |
|---|---|---|---|---|---|---|
| 1 | AGATCTTGAG | CTCGACTGCA | GCTCGGAGAC | AATCATATGG | GAGAAGCAAT | TGGAAGATAG |
| 61 | AAAAAAGGTA | CTCGGTACAT | AAATATATGT | GATTCTGGGT | AGAAGATCGG | TCTGCATTGG |
| 121 | ATGGTGGTAA | CGCATTTTTT | TACACACATT | ACTTGCCTCG | AGCATCAAAT | GGTGGTTATT |
| 181 | CGTGGATCTA | TATCACGTGA | TTTGCTTAAG | AATTGTCGTT | CATGGTGACA | CTTTTAGCTT |
| 241 | TGACATGATT | AAGCTCATCT | CAATTGATGT | TATCTAAAGT | CATTTCAACT | ATCTAAGATG |
| 301 | TGGTTGTGAT | TGGGCCATTT | TGTGAAAGCC | AGTACGCCAG | CGTCAATACA | CTCCCGTCAA |
| 361 | TTAGTTGCAC | CATGTCCACA | AAATCATATA | CCAGTAGAGC | TGAGACTCAT | GCAAGTCCGG |
| 421 | TTGCATCGAA | ACTTTTACGT | TTAATGGATG | AAAAGAAGAC | CAATTTGTGT | GCTTCTCTTG |
| 481 | ACGTTCGTTC | GACTGATGAG | CTATTGAAAC | TTGTTGAAAC | GTTGGGTCCA | TACATTTGCC |
| 541 | TTTTGAAAAC | ACACGTTGAT | ATCTTGGATG | ATTTCAGTTA | TGAGGGTACT | GTCGTTCCAT |
| 601 | TGAAAGCATT | GGCAGAGAAA | TACAAGTTCT | TGATATTTGA | GGACAGAAAA | TTCGCCGATA |
| 661 | TCGGTAACAC | AGTCAAATTA | CAATATACAT | CGGGCGTTTA | CCGTATCGCA | GAATGGTCTG |
| 721 | ATATCACCAA | CGCCCACGGG | GTTACTGGTG | CTGGTATTGT | TGCTGGCTTG | AAACAAGGTG |
| 781 | CGCAAGAGGT | CACCAAAGAA | CCAAGGGGAT | TATTGATGCT | TGCTGAATTG | TCTTCCAAGG |
| 841 | GTTCTCTAGC | ACACGGTGAA | TATACTAAGG | GTACCGTTGA | TATTGCAAAG | AGTGATAAAG |
| 901 | ATTTCGTTAT | TGGGTTCATT | GCTCAGAACG | ATATGGGAGG | AAGAGAAGAA | GGGTTTGATT |
| 961 | GGCTAATCAT | GACCCCAGGT | GTAGGTTTAG | ACGACAAAGG | CGATGCATTG | GGTCAGCAGT |
| 1021 | ACAGAACCGT | CGACGAAGTT | GTAAGTGGTG | GATCAGATAT | CATCATTGTT | GGCAGAGGAC |
| 1081 | TTTTCGCCAA | GGGTAGAGAT | CCTAAGGTTG | AAGGTGAAAG | ATACAGAAAT | GCTGGATGGG |
| 1141 | AAGCGTACCA | AAAGAGAATC | AGCGCTCCCC | ATTAATTATA | CAGGAAACTT | AATAGAACAA |
| 1201 | ATCACATATT | TAATCTAATA | GCCACCTGCA | TTGGCACGGT | GCAACACTCA | CTTCAACTTC |
| 1261 | ATCTTACAAA | AGATCACGTG | ATCTGTTGTA | TTGAACTGAA | AATTTTTTGT | TTGCTTCTCT |
| 1321 | CTCTCTCTTT | CATTATGTGA | GAGTTTAAAA | ACCAGAAACT | ACATCATCGA | AAAAGAGTTT |
| 1381 | AAACCATTAC | AACCATTGCG | ATAAGCCCTC | TCAAACTTCC | TCCAGACTGT | TTGTCATCCA |
| 1441 | ATTGGTAAGA | TTAATTATCA | TATACCCCGG | ATAAGAAAAG | AAGCAAGCAC | ACAATGGATC |
| 1501 | AACTGGCCGG | CCCGAATTTT | CAAAAATTCT | TACTTTTTTT | TTGGATGGAC | GCAAAGAAGT |
| 1561 | TTAATAATCA | TATTACATGG | CATTACCACC | ATATACATAT | CCATATACAT | ATCCATATCT |
| 1621 | AATCTTACTT | ATATGTTGTG | GAAATGTAAA | GAGCCCCATT | ATCTTAGCCT | AAAAAAACCT |
| 1681 | TCTCTTTGGA | ACTTTCAGTA | ATACGCTTAA | CTGCTCATTG | CTATATTGAA | GTACGGATTA |
| 1741 | GAAGCCGCCG | AGCGGGTGAC | AGCCCTCCGA | AGGAAGACTC | TCCTCCGTGC | GTCCTCGTCT |
| 1801 | TCACCGGTCG | CGTTCCTGAA | ACGCAGATGT | GCCTCGCGCC | GCACTGCTCC | GAACAATAAA |
| 1861 | GATTCTACAA | TACTAGCTTT | TATGGTTATG | AAGAGGAAAA | ATTGGCAGTA | ACCTGGCCCC |
| 1921 | ACAAACCTTC | AAATGAACGA | ATCAAATTAA | CAACCATAGG | ATGATAATGC | GATTAGTTTT |
| 1981 | TTAGCCTTAT | TTCTGGGGTA | ATTAATCAGC | GAAGCGATGA | TTTTTGATCT | ATTAACAGAT |
| 2041 | ATATAAATGC | AAAAACTGCA | TAACCACTTT | AACTAATACT | TTCAACATTT | TCGGTTTGTA |
| 2101 | TTACTTCTTA | TTCAAATGTA | ATAAAAGTAT | CAACAAAAAA | TTGTTAATAT | ACCTCTATAC |
| 2161 | TTTAACGTCA | AGGAGAAAAA | ACCCCGGATC | TCATATGCCT | GCAGG | |

In one embodiment the strain has, linked to the centromere of every chromosome, both a galactose-inducible transcription initiation site that destabilizes the centromere and a counterselectable URA3 gene. In one aspect the inventive donor strain solves the problem of undesirable chromosome transfer since galactose grown cells destabilize all of the donor chromosome and those chromosomes are efficiently counterselected on medium containing 5-fluoro-orotic acid (5FOA), a drug that kills Ura3+ cells (Boeke et al. 1987). Additionally, the recipient strain need only contain a ura3 mutation and another recessive marker to accommodate selection for the plasmid being transferred. In one embodiment the recipient strain can be yeast gene disruption library (Winzeler et al. 1999) which fits these criteria. Other embodiments contemplate yeast gene disruption libraries wherein gene disruptions are created in different genetic background, or wherein gene disruptions can be marked with different markers.

In another aspect the invention provides alternative methods of producing conditionally functional centromeres that include, but are not limited to the following embodiments:

i. Alteration of the yeast donor strain centromere binding proteins such that a novel DNA sequence is recognized as a centromere. Coordinate alteration of all 16 donor yeast centromere sequences to this novel sequence results in centromeres that function only in the donor strain and not in any wild-type (recipient) yeast strains. ii. Using a closely related yeast species that has divergent centromere sequences that do not function in Saccharomyces cerevisiae but is still able to initiate mating with Saccharomyces cerevisiae strains.

### Construction of the Universal Donor Strain

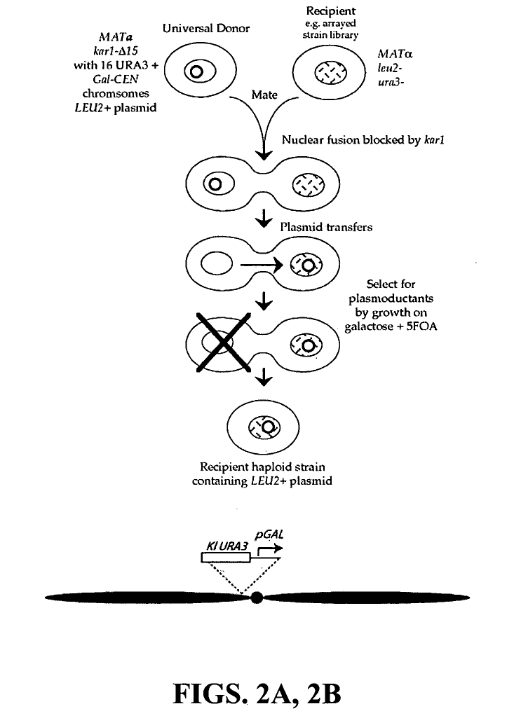

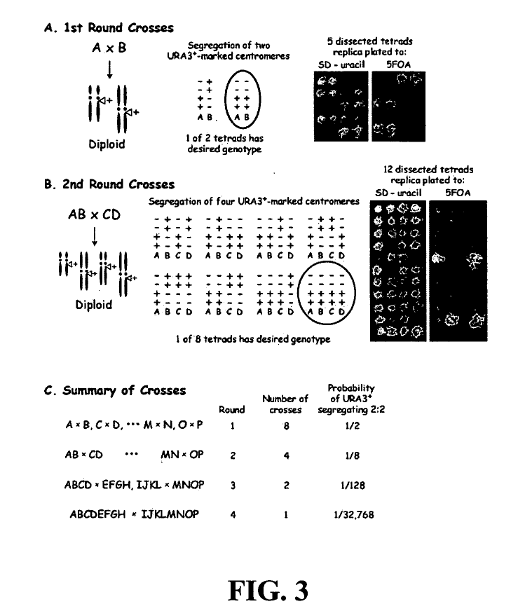

To create the sixteen-tuple URA3 strain, we used a set of URA3-marked chromosomes (FIG. 2). 32 strains (16 MATa, 16 MATalpha) were produced in which a galactose-regulated promoter was introduced upstream of the centromere of a single chromosome. These centromeres were also marked with the URA3 gene from Kluyveromyces lactis as a selectable marker for integration. Using this set of 32 strains, we crossed different marked chromosomes together at first by standard mating and tetrad dissections (FIG. 3). As the URA3 markers are centromere-linked, little or no crossover occurs between the markers generating only ditype tetrads. The desired tetrads in each step of the construction are those where the two spores are Ura+and two are ura−. In the first round of crosses, 50% of the tetrads are parental (4 Ura+) and 50% are nonparental (2 Ura+: 2 ura−) in which the Ura+ strains contain both marked chromosomes (FIG. 3a). In the second round, strains containing 2 URA3+ marked chromosomes were mated to produce a strain with 4 different marked chromosomes (FIG. 3b). In this case 1 in 8 tetrads, on average, segregate 2:2 for uracil prototrophy.

In the 3rd and 4th rounds of crosses (4×4 and 8×8), the probability of obtaining the correct 2:2 tetrad decreases to 1 in 128 and 1 in 32,768 tetrads respectively. For these crosses we performed a tetrad-based screen to identify the correct segregants. In these cases, the multiply URA3+ marked strains were crossed to generate diploids heterozygous for the URA3 chromosomes. The resultant diploid strain was sporulated and the tetrads were diluted and plated onto rich media (YPD) to generate one original colony from each tetrad, wherein the spores from a single tetrad grow adjacent to each other. A tetrad contains the four haploid meiotic products (spores) in a single ascus. Therefore, these colonies were a mix of MATa and MATalpha haploid segregants as well as diploids produced by mating of those haploids. The desired rare 2:2 tetrads have all of the URA3 marked chromosomes segregating together in two spores while the other two spores are uracil auxotrophs and therefore also 5FOA resistant. Thus, after replicating onto 5FOA medium, the rare desired tetrad gives rise to a patch of 5FOA resistant cells. Such a colony is mixed as it also contains the two desired spores with all of the chromosomes marked with URA3. To isolate these spore clones, the 5FOA resistant colonies from the original YPD plate were restreaked. The restreaked single colonies represent cells from each of the four spores of the original tetrad plated on YPD. Those single colonies were next tested by standard means to find the URA3+ haploids that contained all of the marked chromosomes in a single strain. For example, for the final round of crosses (8×8), over 130,000 tetrads were plated and nine 5FOA resistant colonies were isolated. From these nine, two produced a mix of URA+ and ura− colonies upon restreaking from the YPD plate. To confirm that the chromosome complement was as predicted, we analyzed these cells by PCR as described in FIG. 4A. As shown in FIG. 4B, these strains contain all 16 marked chromosomes. We also performed a second PCR to show that no wild-type CEN sequences were present in these strains (FIG. 4C). Thus the observed number of tetrads containing 2:2 segregation for URA3 (≈ 1/65,000) was within two-fold of the expected number ( 1/32,768).

To estimate the meiotic chromosome stability of the marked strains, we generated homozygous diploids containing all 32 URA3-marked chromosomes. These diploids were sporulated and tetrads were dissected to determine the spore viability. Of 92 dissected spores, 87 gave all four viable spores (95% viability), which is similar to the wild-type parental W303 strain. The homozygous diploid strain also has the same growth rate as the parental strain indicating that introduction of multiple centromere proximal markers does not have a negative effect on growth. Finally, the kar1Δ15 mutation (Vallen et al. 1992) was introduced using our standard allele replacement method (Erdeniz et al. 1997) to produce the final donor strain.

### Recipient Strain

In another aspect the inventive donor strain solves the problem of complex genetic manipulations to create a recipient strain that is suitable for use in kar-mediated transfer of genetic material. The most important consideration for the recipient strain for use with the donor strain of the invention is that the recipient strain lacks the counterselectable marker of the centromeric counterselectable cassette. The genetic background of the recipient strain also allows for destabilization of the conditional centromere of the counterselectable cassette of the donor chromosome. Furthermore, the recipient strain lacks an endogenous or exogenous selectable marker which leads to a metabolic deficiency or drug sensitivity. In one embodiment the recipient is auxotrophic for the selectable marker borne on the extrachromosomal genetic material. This marker configuration allows for the selection of recipient cells that have acquired the extrachromosomal genetic material while selecting against the donor chromosomes marked with a counterselectable cassette which comprises a counterselectable marker and a conditional centromere. In one embodiment, the donor strain chromosomes have the K. Lactis URA3 gene as a counterselectable marker while the recipient strain is auxotroph for uracil, i.e. the strain is ura−. In another embodiment, the recipient strain is ura− and GAL+. Finally the recipient and donor strains must be of a mating type that allows them to initiate mating.

### Genetic Material

Extrachromosomal genetic material that can be transferred between strains by the method of the invention can be circular DNA, for example but not limited to circular plasmid DNA, or linear DNA, for example but not limited to YACs or yeast chromosomes. The most important consideration is that the extrachromosomal genetic material has a selectable marker that allows for its selection. In one embodiment the selectable marker of the extrachromosomal DNA complements the auxotrophy of the recipient or donor strain caused by a mutation in the selectable marker in the recipient strain or a mutation in the selectable marker in the universal donor strain. It is clear to a person skilled in the art that certain markers such as URA3, TRP1 or LYS5 can be used both as selectable and counterselectable markers. Therefore the counterselectable marker of the centromeric cassette is different from the selectable marker of the extrachromosomal genetic material.

The extrachromosomal genetic material may encode one or more nucleotide sequences of interest. The nucleotide sequences can encode a gene of interest, genetic assays, target nucleotide sequences or any other nucleotide sequence of interest. The gene or genes of interest is operably linked to a promoter which can be a regulatable promoter, for example but not limited to GAL (galactose induced promoter), CUP1 (copper induced promoter), MET15 (methionine regulated promoter). In some embodiments the regulatable promoter of the gene of interest may be the same or may be different from the regulatable promoter of the conditional centromere.

Alternatively the selectable marker on the extrachromosomal genetic material can be a gene whose product confers drug resistance to the cells. A non-limiting example is the heterologous drug resistance marker KanMX which confers kanamycin resistance.

Another aspect of the invention allows for the selected transfer of desired chromosomal material. In one embodiment, the desired chromosomal material which is transferred from the donor to the recipient is a yeast chromosome of choice from the donor strain. It this embodiment the yeast chromosome of choice is not marked with a counterselectable cassette. The yeast chromosome of choice is marked with a selectable marker which complements a metabolic deficiency or drug sensitivity of the recipient strain.

### Transfer of Extrachromosomal Genetic Material Using the Universal Donor Strain

The donor strain was tested for its performance in plasmoduction. In one embodiment the strain was transformed with a LEU2-marked CEN plasmid (pRS415) and propagated it on plates as a lawn of yeast cells. Wild-type W303 strains and strains from the gene disruption library were grown as patches on a second plate. Matings were performed by replica plating the strains together on rich medium for 6 hours then replica plating to various selection plates. Selection for plasmoductants was performed simultaneously on synthetic galactose plates lacking leucine (Sgal-leu) and Sgal-leu plates that also contained 5FOA (Sgal-leu+5FOA). As shown in FIG. 5, several hundred papillae form on the Sgal-leu plates where the MATalpha strains came into contact with the MATa donor lawn. No colonies were observed at the position of the MATa patch indicating that these papillae require mating fusion to appear. After directly testing the mating mixture on Sgal-leu+5FOA plates, no colonies appeared. However, after replica plating the colonies that grew on the Sgal-leu to a Sgal-leu+5FOA plate, several hundred colonies per patch were found. This delay in the “expression” of 5FOA resistance is due to the need for approximately 3 generations to dilute out the Ura3+ gene product (Ronne and Rothstein 1988) that was introduced when the heterokaryon formed. In summary, the galactose-induced centromere destabilization works well as the first selection against the donor nucleus as it is donor chromosome specific and does not affect the ability of the heterokaryon to produce a haploid bud containing the recipient nucleus. Approximately 3 generations after the recipient nucleus buds, the Ura3 gene product is sufficiently diluted for 5FOA resistant haploids to form. In other embodiments, the invention contemplates selections which may not require three generations of growth before selection is applied.

In another embodiment the universal donor stain can be transformed to carry other extrachromosomal genetic material, including but not limited to 2-micron based plasmids, YACs, etc. In yet another embodiment, a gene of interest encoded on the extrachromosomal DNA can be operably linked to regulatory element which can be inducible, repressible or constitutively expressed.

The invention also contemplates the transfer of multiple distinct units of extrachromosomal genetic material, for example multiple plasmids having different selectable markers, from one donor into multiple recipients. Such transfer is possible as long as the distinct units have different markers and the recipient strain lacks these markers permitting the selection of the transferred genetic material. The invention also contemplates multiple rounds of transfer of extrachromosomal material provided that the extrachromosomal material has different markers and the recipient strain lacks these markers permitting the selection of the transferred genetic material.

The background level of rare diploid formation was monitored by determining the fraction of papillae that are able to mate. Diploid formation was low on Sgal-leu plates (<1%) and was virtually eliminated by the sequential selection scheme (Sgal-leu to Sgal-leu+5FOA; data not shown). All current experiments can use the sequential selection strategy. The recovered colonies after sequential selection were all MATalpha indicating that only the recipient strain was recovered.

To assess the background level of co-transfer of chromosomes along with the desired plasmid, a subset of strains was chosen from the MATalpha deletion library to be auxotrophs for metabolic genes that are correspondingly wild-type in the W303 donor strain. We tested plasmoductants resulting from these crosses to determine if the wild-type allele from the W303 donor strain ever co-transferred into the library strain. FIG. 6 shows arg4 and arg5/6 deletion library auxotrophs and a W303 strain as a control with plasmoductant colonies on successive replicas after a 6 hr mating. The top row shows the initial Sgal-leu replica plate, the middle row shows the subsequent Sgal-leu+5FOA plate (which was replica plated from the top row plate two days earlier) and the bottom row shows the final SD-leu-arg plate (which was replicated from the middle row plate three days earlier). Since the W303 strain itself is ARG+, all of the plasmoductants grow on the double dropout media. Analysis of the arg4 and the arg5/6 strains shows that no allele transfer of ARG4 or ARG5/6 from W303 occurred at the same time as plasmid transfer. Several other library auxotrophs were also tested and a summary of the results is shown in Table 2. In all cases examined, the frequency of allele co-transfer is less than 0.16% and in most cases even lower.

There are at least two ways that an allele from W303 can co-transfer with the plasmid. One is by transfer of the W303 chromosome containing the wild-type copy of the auxotrophic marker. The other way is via gene conversion where only the genetic information for the gene transfers. Our initial analyses of the prototrophic papillae recovered in these experiments suggest that they result from gene conversion rather than transfer of a complete chromosome sequence. Thus the frequency of chromosome transfer may be even lower than our genetic analysis suggests.

TABLE 2
Frequency of spurious allele transfer during
plasmoduction for 8 yeast genes.

| allele | total papillae | auxotrophs | percent |
|---|---|---|---|
| ade1 | 2489 | 0 | <0.04 |
| arg4 | 2048 | 0 | <0.05 |
| arg5/6 | 1319 | 1 | 0.08 |
| arg1 | 635 | 0 | <0.2 |
| arg3 | 650 | 0 | <0.2 |
| tyr1 | 1279 | 0 | <0.08 |
| ade3 | 2095 | 2 | 0.1 |
| trp4 | 1244 | 2 | 0.16 |

Eight different gene disruption strains were used as in FIG. 6 to determine the frequency of spurious allele transfer during plasmoduction. Auxotrophs column indicates the number of colonies from the LEU+ papillae that grew on the double dropout replicas. Percent figures preceded by a < are estimates of an upper bound of the percentage calculated as 1/total plasmoductants.

The invention also contemplates using the universal donor strain to transfer multiple distinct copies of extrachromosomal genetic material having the same selectable marker into a recipient strain (many into 1) (Georgieva and Rothstein, 2002).

### Transfer of Cytoplasmic Material

In another aspect, the invention allows for directional transfer of cytoplasmic material while preventing spurious chromosome transfer from the donor strain.

In one embodiment, a plasmid containing a selectable marker, which allows for its introduction into the yeast gene disruption library, also comprises a nucleic acid which encodes an ochre suppressible marker, for example but not limited to lacZ. The plasmid with the ochre suppressible marker is introduced into the universal donor strain, which has been made [PSI+] by overexpression of SUP35. Overexpression of SUP35 induces formation of the yeast prion (Psi). The overexpression plasmid for SUP35 is lost from the donor strain before the donor is mated with the existing library strains. Most recipients are blue due to cytoplasmic transmission of Psi and subsequent suppression of the lacZ sequence. Recipient stains that remain white are of interest as they are indicative of mutations in genes that interfere with Psi+ propogation. Such genes can be potential drug targets for inactivation in diseases associated with prion formation.

In another embodiment the recipient library strain is ade2-1. ade2-1 is an ochre suppressible mutation. In the absence of ochre suppression, the ade2-1 mutation leads to accumulation of red pigment. ade2-1 cells are white in the presence of suppression. After fusion and cytoplasmic mixing, most of the colonies are white due to transfer of Psi+. Recipient strains which are unable to maintain the Psi+ prion state remain red. Recipient stains that remain red are of interest as they are indicative of mutations in genes that interfere with Psi+ propagation. Such genes can be potential drug targets for inactivation in diseases associated with prion formation

### Automation of the Method

To increase the reliability of genome-wide screens, decrease costs, and increase throughput; the invention provides for automating key features of the screening procedure.

To automate our screens, we have re-array the gene disruption library into 384-well plates in quadruplicate samples. This increases the density of the strains per plate for mating and subsequent selections. Although it would be helpful for automation to do the matings in liquid medium, we have found that mating between two yeast strains takes place most efficiently on solid medium (Arthur 1991). Therefore, we will transfer 384 strains at a time from liquid media onto rectangular Omniplates (Nunc) containing YPD agar by using a 384 pin replicating tool. 1.58 mm diameter slotted pins can be used to reproducibly deliver 0.5-5 μl volumes of a yeast culture depending on the size of the slot. In one version of the experiment, the transfer of the disruption strain will be followed by transfer of the plasmid donor culture at the same positions using the pin tool. The deletion library can be transferred to a plate containing a freshly spread lawn of the plasmid donor strain obviating the need to pin the donor strain onto the mating plate. After mating, the colonies will be transferred by pin tool to another rectangular plate for selection. For this operation, solid pins are appropriate for this plate-to-plate transfer.

Automation of these steps is possible. We have successfully used hand pin tools for such transfers in preliminary experiments, however a robotic device such as the V&P Scientific Pin Tool Robot or the Singer Instruments RoTor will be more accurate and reproducible over many experiments. Automation will also keep the cost of these experiments relatively low. Once the gene disruption library is re-arrayed to 384 well plates it becomes a stock that can be used in many such experiments using the pin tools to transfer volumes of culture eliminating the expense of using disposable pipette tips for similar operations.

To fully evaluate our method, we will screen both the MATa and MATalpha versions of the gene disruption library with the TOP1-T₇₂₂A donor plasmid. We expect the same results from the MATa and MATalpha libraries. Any differences between the results from each library will be documented and the identifying sequence tags (Winzeler et al. 1999) from any such strains involved will be compared to the database of tag sequences maintained by the deletion consortium. Bookkeeping errors may account for differences in the data sets, for example, we have determined that the rad51 strain from the MATalpha library is mislabeled.

Once we have optimized our method for large-scale screening, we will direct our attention to other mutant topoisomerases. In addition to the TOP1-T₇₂₂A screen just described, we will use a TOP1-Y₇₂₇F mutant, which disrupts the active site tyrosine in Top1. Galactose-induced overexpression of this mutant from a 2μ based vector is lethal in some strain backgrounds and the underlying mechanism for this lethality is not clear. We suspect that the identification of SDL interactions with this allele will provide insight on the genetic pathway(s) that is required to deal with non-functional Top1 proteins.

### Data Handling

Data generated from the screens performed will be organized into a database. Scans of the petri plates will be organized so that a plate image is searchable by the name of each of the gene disruptions on that plate to provide access to raw data from the screens. In addition, data from each screen will be summarized in table form. In most cases this will exist as binary designations, i.e. sensitive or insensitive to Top3-Y₃₅₅F overexpression. However, we have already shown that we can identify graded responses to Top1-T₇₂₂A overexpression, thus the data set will be recorded as having two sensitivity levels.

Results of the SDL screens will be compared to other genomic datasets by making use of clustering algorithms (Liu and Califano 2003). Such comparisons will help define the relationship of the individual reference alleles to existing genetic networks.

### EXAMPLES

The Saccharomyces gene disruption library is an important resource for the study of gene function. This library has been used to identify gene disruptions resulting in sensitivity to chemicals, drugs and other external environmental stresses (Winzeler et al. 1999; Parsons et al. 2004). The library has also been used to identify synthetic lethal genetic interactions by systematically crossing a reference mutation to every gene disruption strain, sporulating the diploid and screening for lethality of the double mutant progeny (Tong et al. 2001). What has been missing thus far is an efficient method to transfer plasmids into the entire library. High efficiency lithium acetate-based transformations require several manipulations. Furthermore, when transformation is scaled down, it becomes inefficient making it impractical for large numbers of samples. Transmission of plasmids to meiotic segregants is tedious and inefficient, so mating and sporulation is not an ideal way to introduce plasmids into the gene disruption strains. The universal donor strain of the invention can serve as a plasmid transfer vehicle for use with the gene disruption library. Plasmids are transferred during an abortive mating between the donor and the recipient cell due to a kar1 mutation in the donor strain.

In one aspect the invention is a new Saccharomyces cerevisiae strain that can be used to transfer plasmids into any ura3-budding yeast strain, a process we term plasmoduction. This universal plasmid donor strain provides an important resource that has broad applications in yeast genetics and drug discovery. The procedure used is based on mating and is performed by replica plating of mixtures of donor and recipient strains. The method of the invention is particularly suited to the transfer of plasmids into a library of yeast strains such as the set of haploid gene disruptions. Using the stain of the invention, mating becomes an efficient means to transfer plasmids to the library, obviating the need to perform DNA transformation on each member of the library. The strain and the method of the invention were used to screen the Saccharomyces gene disruption library for synthetic dosage lethality (SDL) induced by overexpressing a gene product. SDL is defined by the situation where overexpression of a gene results in lethality in certain mutant backgrounds. The set of mutations uncovered by these types of screens have been particularly successful in defining genetic interactions of members of cellular complexes. The networks defined in this way have great potential to expand our knowledge of genetic interactions in yeast. The experimental approaches outlined here will serve as paradigms for the development and use of similar methods in other organisms as gene disruption libraries become available. The screens use plasmid-based overexpression of either a wild-type protein or catalytic mutant alleles of a gene.

The potential applications for this type of plasmid transfer are numerous given the number of plasmid-borne reporter systems used by yeast biologists. However, screens of a strain library are most easily performed using a simple readout. One type of experiment would be to transfer a gene fused to a green fluorescent protein (GFP) or a beta-galactosidase reporter to study protein levels in all the gene disruption strains. An even simpler readout would be to look for gene disruptions that cause lethality or poor growth in the presence of an overexpressed gene.

There are two general types of genetic interactions that can be determined from an overexpression screen. One type of genetic interaction can be identified by overexpression of a wild-type gene that is part of a multiprotein complex and whose overexpression does not result in lethality in an otherwise wild-type strain. If another member of the multiprotein complex is mutated, the overexpression of the wild-type gene causes an imbalance in the cellular complex that results in cell death. This type of genetic screen is often referred to as synthetic dosage lethality (SDL) and has been used to identify interactions among components of the kinetochore as well as interactions between CTF18—a clamp loader homolog involved in sister chromatid cohesion—and DNA replication genes (reviewed in Measday and Hieter 2002, also see Hanna et al. 2001; Measday et al. 2002).

Another type of interaction can be identified when overexpression of a mutant gene results in lethality. This screen can identify genes that respond to, or prevent the “lesion” caused by expression of the reference gene. Data for this type of screen in which expression of a TOP1 mutant results in a low level of DNA lesions is shown in Example 1. Strains that are DNA repair or checkpoint deficient result in increased sensitivity to expression of this TOP1 mutant and grow poorly (Reid et al. 1999; Fiorani et al. 2004). Similarly, overexpression of a catalytic mutant of an enzyme can produce a nonfunctional enzyme that is capable of forming complexes with its normal binding partners in the cell, but cannot carry out a catalytic function. Overexpression of such a mutant can titrate out the function of a wild-type allele and effectively produce a null phenotype. Introducing an overexpressed catalytic mutant into a gene disruption library can then identify the same types of genetic interactions as seen when screening for synthetic lethal interactions using a null mutant. We show that overexpression of a catalytic mutant of DNA topoisomerase III (top3-Y₃₆₅F) produces a null-like phenotype.

The system that we describe is flexible allowing the regulation of expression levels. Thus, expression levels can be titrated such that the wild-type strain can tolerate some overexpression of the gene of interest, while specific mutants cannot.

The inventive plasmoduction method greatly extends the capability of performing these screens in the context of a gene disruption library—or any other strain library. We propose to apply this new method on a genomic scale to select for interacting genes by screening with two important classes of proteins, DNA topoisomerases and DNA helicases. Topoisomerases and helicases are sufficiently well characterized enzymes to allow us to make specific catalytic mutations (Y—F mutants for the topoisomerases and K—R/K-A mutations in the ATPase domains of helicases). DNA toposiomerases are important in nearly every aspect of DNA metabolism. These enzymes allow DNA to be transcribed, replicated, condensed, segregated and repaired despite the “knotty” topological problems inherent to DNA structure. Each of these enzymes catalyzes a break in one or both strands of the double helix by a trans-esterification reaction in which an active site tyrosine is transiently covalently bound to the phosphate backbone (reviewed in Hardy et al. 2004). DNA topoisomerase I cleaves a single DNA strand and allows rotation of the cleaved strand before religation to relax both positive and negative supercoils (reviewed in Wang 1996). Although not essential in yeast, Top1 is the major relaxation activity during DNA replication (Kim and Wang 1989). DNA topoisomerase II (Top2) cleaves both DNA strands and passes a duplex through that transient break. This not only relieves supercoiling, but is also the principle method by which chromosomes are decatenated prior to anaphase (reviewed in Ullsperger et al. 1995). DNA topoisomerase III (Top3) cleaves a single DNA strand with a preference for single-stranded or highly underwound DNA (Kim and Wang 1992). Top3 is also nonessential in yeast, however, mutants are slow growing due to unregulated activity of the interacting DNA helicase SGS1 (Wallis et al. 1989; Gangloff et al. 1994).

Top1 and Top2 are also important cellular targets of chemotherapy drugs due to their roles in cell proliferation (reviewed in Denny 2004). Camptothecin and its derivatives specifically target the covalent complex of Top1 bound to DNA and inhibit religation of the DNA backbone. Unrepaired covalent complexes are converted to double-stranded DNA breaks (DSBs) during replication resulting in apoptosis in vertebrates and cell death in yeast (Nitiss and Wang 1988; Pommier et al. 1994). Top2 is the cellular target of the epipodophylotoxins. These drugs also inhibit religation of the covalently bound DNA and result in DNA lesions and cell death (reviewed in Wang 1994). TOP1 mutations have also been described that result in a stabilization of the Top1-DNA covalent intermediate in the absence of drug and mimic camptothecin-induced lethality upon overexpression (Megonigal et al. 1997). Reduced levels of expression of these alleles are tolerated in wild-type cells, but cause death in repair-deficient strains (Reid et al. 1999; Fiorani et al. 2004).

Helicases catalyze ATP-dependent unwinding of DNA and mobilization of DNA-protein complexes. The paradigm for a DNA helicase is bacterial dnaB gene, which is the main replicative helicase for the bacterial replication fork. This enzyme binds to a 5′ single strand DNA end and hydrolyses ATP as it unwinds duplex DNA in a 5′ to 3′ direction during replication. In yeast, many different helicases take on specialized roles in DNA replication and repair associated with replication. For instance, the replicative helicase is most likely composed of a heterohexamer of Mcm2-7 each of which has only marginal helicase activity in vitro, but seems to be in the right place at the right time in vivo while Dna2 appears to play a major role in lagging strand replication (Reviewed in Labib and Diffley 2001). Problems during replication such as fork restart or replication through specialized structures require other helicases. The recQ helicases such as SGS1 are important for replication restart after fork collapse (Cobb et al. 2003). Pif1 and Rrm3 helicases affect replication in the condensed DNA of the nucleolus (Bessler et al. 2001). Other helicases play direct roles in recombination such as Rad54 and Rdh54 (Klein 1997). Our efforts will focus on overexpression screens using a small subset of helicases that are involved in recombination (Rad54/Rdh54/Srs2), genome stability (Sgs1/Pif1/Rrm3) and replication (Mcm2-7).

SDL screens with wild-type and mutant alleles of topoisomerases and helicases will complement the current genomic approaches for identifying genetic interactions such as the synthetic genetic array (SGA) (Tong et al. 2001; Jorgensen et al. 2002). An advantage of the SDL approach is the added flexibility to use an essential gene as the overexpressed allele. In principle, essential genes can be used in the standard SGA approach, however, to maintain viability in haploid cells, the gene would have to be a conditional allele and would also need to be linked to a selectable marker to select for cosegregation with the library disruption. In contrast, the reference essential gene for our new plasmoduction-based SDL screen is simply an overexpressed wild-type allele.

This invention is illustrated in the Examples that follow. These sections are set forth to aid in an understanding of the invention but are not intended to, and should not be construed to, limit in any way the invention as set forth in the claims which follow thereafter.

### Example 1

### Transfer of Plasmid Permitting the Regulated Expression of a DNA Topoisomerase I Protein Mutant

This example illustrates a screen of the gene disruption library with a catalytic mutant of DNA toposiomerase I. Mutation of Thr 722 to Ala shifts the catalytic equilibrium of Top1 toward the covalent enzyme-DNA intermediate mimicking the action of the chemotherapy drug camptothecin (Megonigal et al. 1997). Overexpression of TOP1-T₇₂₂A results in cell death while low level expression is tolerated. However, in DNA repair deficient strains, even low levels TOP1-T₇₂₂A expression results in cell death (Reid et al. 1999). Several classes of genes affect sensitivity to camptothecin and TOP1-T₇₂₂A expression. These include DNA repair genes, lagging strand replication genes, and DNA damage checkpoint genes. Thus screening for genes affecting Top1-T₇₂₂A expression in the deletion library can be used to compare the ability of this new screen to find genes already known to affect topoisomerases as well as to find new ones.

In our initial attempts at plasmoduction with the Top1-T₇₂₂A mutant on a 2μ vector, we found that very high overexpression of the mutant negatively affects the plasmoduction process. As an alternative and to allow regulated gene expression, we cloned the Top1-T₇₂₂A gene under a CUP1 promoter so that expression could be induced after plasmoduction. Induction of the CUP1 promoter by copper sulfate is graded with maximal expression at approximately 100 μM copper. At lower concentrations, expression falls off roughly linearly (Butt et al. 1984; Karin et al. 1984). FIG. 7A shows the results of a LiOAc transformation of the pCUP-Top1-T₇₂₂A mutant or a wild-type pCUP-Top1 plasmid into several strain backgrounds in the absence of copper. A wild-type and rad9 strain were efficiently transformed with this plasmid, however no transformants were recovered from the rad50 strain suggesting that even the low level of expression from the CUP1 promoter in the absence of induction is synthetically lethal in a rad50 mutant background. As shown in FIG. 7B, addition of copper changes viability. In wild-type strains, expression Top1-T₇₂₂A is tolerated up to 20 μM copper, but induces lethality at 100 μM. In contrast rad9 strains show some sensitivity at 4 μM and a 100-fold reduction in viability at 20 μM copper compared to the 0 μM controls (FIG. 7B). These experiments show that the CUP1 promoter can be used efficiently to regulate the expression of genes for use in our screen.

The graded response to copper can also be observed in the expression of wild-type TOP1 on plates containing camptothecin. The library strain is TOP1 and relatively low doses of drug do not affect viability. However, increasing the level of expression of TOP1 increases camptothecin sensitivity by increasing the amount of the drug target (FIG. 7B, last row). This shows that overexpression of a drug target gene in the presence of the drug can also be used to search for synthetic interactions.

Plasmid transfer via plasmoduction was tested using a series of “semi-random” library strains. Six strains were chosen from four rows of the library, each row of which contained a known gene that interacts with camptothecin/Top1 (FIG. 8A). The pCUP-Top1, pCUP-Top1-T₇₂₂A and a vector control plasmid were transformed via the LiOAc method into the plasmoduction donor strain and the transformants were used for plasmid transfer into 24 strains described above. As before, lawns were made for the donor strains and crosses were performed to patches of the 24 library strains by replica transfer. FIG. 8B shows the results of selection for plasmoductants after two rounds (as described above) in the absence of copper induction. Whereas the vector control and the wild-type TOP1 plasmids can be transferred into each of the library strains, the low levels of background expression of Top1-T₇₂₂A clearly does not permit the recovery of plasmoductants into the rad50 and rad52 mutants. Next, serial dilutions of plasmoductants from each strain were grown overnight and spotted onto medium containing 20 μM copper. This further screening of the plasmoductants showed that rad27 and rad9 strains also showed reduced viability upon increased TOP1-T₇₂₂A expression (FIG. 8C). Thus two levels of sensitivity can be clearly determined in this screen.

The following example demonstrated that the plasmid transfer method is robust. In this example, a plasmid permitting the regulated expression of a mutant (Top1-T₇₂₂A) was transferred into a subset of strains from the yeast gene disruption library and the SDL strains were identified. The screen can be completed for the complete set of strains from the yeast gene disruption library.

The above example using overexpression of the TOP1-T₇₂₂A mutant to screen the yeast gene disruption library is an important proof of principle for the plasmoduction screens we want to do. We completed the screen of the entire haploid gene disruption library using the TOP1-T₇₂₂A mutant. In this screen 4827 strains were tested and 141 (2.9%) of the library strains were found to be sensitive to TOP1-T₇₂₂A expression. The following genes when mutated can be sensitive to TOP1-T₇₂₂A expression can be classified in the following functional categories:

DNA Repair: SAE2, MRE11, XRS2, RAD50, RAD52, RAD59, RAD55, RAD57, RAD51, RAD54, MMS4, MUS81, MMS1, NCE4, RTT101, RTT107, RTT109, SRS2, RAD27, HUR1, POL32, MRC1,

Checkpoint: RAD17, CLN3, WHI4,

Nuclear Pore: NUP84, NUP170, NUP60, NUP188,

Chromosome Maintenance: Sister Chromatid Cohesion-CTF4, CTF8, CTF18, DCC1, CSM3, TOF1, Outer Kinetochore-MCM16, MCM21, CHL4, Centromere Binding-CBF1,

Chromatin Remodeling: SAGA HAT Complex, SPT8, RPD3/SIN3/HDAC, SDS3, RPD3, SAP30, NuA4 HAT-EAF7, VID21, HMO1, H2B K123 Ubiquitination-RAD6, LGE1, Other Chromatin Remodeling-ASF1, SWI6, SNF12, CST6,

Mitochondria: GGC1, SHM1, HFA1, MTM1, MEF1, MRPL22, CCR4-NOT Complex-CCR4, POP2, DHH1,

Vesicle Traffic: Localized to Golgi-PMR1, GVP36, VAN1, SAC1, RAV2, Late Golgi to Prevacuole Compartment Sorting-VPS1, VPS8, VPS9, PVC Maturation-BSD2, ATG14, VPS27, VPS23, VPS28, VPS25, VPS4, VPS36, VPS24, VPS20, VPS2, DOA4, PVC to Vacuole-VAM10,

RNA Metabolism/Ribosome: SNR17-A, CDC40, APQ12, NAM7, NOP12, DIA4, SKI3, RPL2B, RPL40A, RPS6A, PAT1, RNY1, BUD31, LSM6, SSZ1, ZOU1,

Cytoskeleton: SHE4, RVS161, RVS167,

Categorization not yet complete: DIA2, YCRO51, NAT3, SPA2, CLG8, YNRO68, NBP2, DEP1, SAG1, PAC10, CIN8, AKR1, YML036, YML095C, YOR364W, YJL046W, YBR099C, HIT1, HIS5, ALG8

The TOP1-T₇₂₂A mutant was chosen for a number of reasons. First, there is specific interest in camptothecins as chemotherapy agents and analysis of the genetic determinants of sensitivity in yeast may ultimately be of use in the clinic. Second, a screen of the gene disruption library against camptothecin has been performed and a number of genes that affect drug sensitivity were identified (Parsons et al. 2004). The dataset produced in this example was compared to the dataset from the drug-based study (FIG. 9). A subset of the mutations identified in the screen were similar to mutations identified in previous screens thus validating the SDL method. Identification of different mutations was informative for two reasons. First, the effects of the Top1-T₇₂₂A mutation were subtly different than the drug and it is possible that different gene disruptions will be identified. Second, our screening procedure could produced “hits” such a transcription factor that affects expression from the CUP1 promoter. Thus differences in sensitivity profiles between the two methods allowed us to assess the sensitivity and reliability of our method while we are optimizing it. Some of the genes identified in this screen are potential drug targets to develop compounds that will selectively kill cells in conjunction with camptothecin (CPT). This may help lower CPT concentrations and limit side-effects from toxicity to high doses of drug. To validate the yeast results in mammalian cells, human cells in particular, homologues of the genes identified in the yeast screens will be silenced using RNAi methods and tested for their sensitivity to CPT. Synthetic dosage lethality confirmed in mammalian cells will allow these genes to become targets for compounds to augment CPT killing of cancer cells.

### Example 2

### DNA Topoisomerase II

The TOP1-T₇₂₂A mutant discussed above is a specific allele that introduces lesions in the cell by altering catalytic function of the enzyme. Another type of allele that can be screened by overexpression is a catalytically dead allele. We have generated the top3-Y₃₅₅F catalytic mutant to examine its affect when overexpressed in yeast. The Y to F alteration was constructed by PCR using specific primers to introduce the mutant sequence, which was subsequently cloned under the control of the pGAL1 promoter for inducible high-level expression. The galactose-inducible mutant, the wild-type allele and a vector control were transformed into a diploid W303 strain and streaked onto plates containing galactose (FIG. 9). Overexpression of the top3-Y₃₅₅F mutant confers severe slow growth in the diploid similar to a homozygous null top3 mutation. Just like a top3 mutant strain, the slow growth of the overexpressed Y to F mutant is suppressed by mutation of sgs1 as well as by mutation of several recombination genes. Thus, the net effect of top3-Y355F overexpression is to confer a “null”-like phenotype on a wild-type cell. This allele cloned under the CUP1 can be used in a SDL screen.

### Example 3

### DNA Topoisomerases II

To evaluate the automation of the method, the plasmoduction screen will be extended to include alleles of DNA topoisomerases II & II. For Top2, we will produce a top2-Y₇₉₃F mutation (Worland and Wang 1989) for these screens. We will evaluate the effect of the top2-Y₇₉₃F mutant expression in a wild-type strain background. Since TOP2 is essential, it is possible that overexpression of this allele will produce a null phenotype. In this case, we will titrate the expression level via copper concentration in the medium to produce a sub-lethal expression level. Strains expressing the top2-Y₇₉₃F mutant will be evaluated for growth rate, sensitivity to topoisomerase II specific drugs, and rDNA recombination frequency in order to determine if sub-lethal expression has a cellular effect. Similar to TOP1 and TOP2 alleles, we will screen both wild-type and the catalytic top3-Y₃₅₅F mutant. As demonstrated in the preliminary results, it appears that top3-Y₃₅₅F expression produces a null-like phenotype and will be useful to find interacting genes in a SDL screen.

We will consider incorporating other topoisomerase mutants into our screen. For instance, recently a mutation in the human TOP2a coding sequence was shown to be lethal in yeast rad52 mutants but not in wild-type strains (Walker et al. 2004). An SDL screen will determine what genetic pathway(s) are involved in this mutant allele.

The examples described herein demonstrate the many potential applications of the universal donor strain of the invention. The main advantage is the technology is simple enough so that any laboratory could perform the operations necessary to screen for mutants affecting a particular gene. In principle, artificial chromosomes containing complicated assays such as silenced chromatin or genetic recombination assays could be transferred into the gene disruption library. In addition, this method does not limit the type of genes that can be tested. For example, in addition to what has been proposed for protein-encoding genes, one could determine SDL when non-protein coding RNA species are overexpressed (e.g., tRNA genes) as well as asking what kinds of mutants are affected when certain DNA sequences are over-represented (e.g., regulatory sequences or telomere sequences cloned onto a 2μ circle vector).

### Example 4

### Identification of Potential Target Genes that when Inhibited will Selectively Kill Cells which Over-Express Specific Genes

Lung cancer is the second most common cancer and the number-one cause of cancer death in both men and women in the US, and hRAD9 has been shown to be over-expressed in 33% of non-small cell lung carcinomas (NSCLC) (Dollinger 2002; Maniwa, Yoshimura et al. 2005). The strain and method of the invention cab be used to dentify potential target genes that when inhibited will selectively kill cancer cells in which members of the Rad9/Hus1/Rad1 (9-1-1) are over-expressed (e.g., NSCLC).

Cells evolved DNA repair mechanisms to prevent tumorigenic mutations and maintain genomic stability. DNA double strand breaks arise from DNA replication and DNA damaging agents such as alkylators, radiation, and replication inhibitors (Melo and Toczyski 2002). Damage-sensing proteins are involved in activating cell cycle checkpoints and recruiting DNA repair proteins to the site(s) of the lesion (Zhou and Elledge 2000). Disruption of checkpoint signaling processes is associated with increased sensitivity to DNA damaging agents, increased genomic instability, and tumorigenesis (Hartwell and Kastan 1994; Bartkova, Lukas et al. 1997). The DNA damage response clamp loading complex, Rad17/RFC2-5, and sliding clamp, Rad9/Hus1/Rad1 (9-1-1), are damage sensors that are recruited to stalled replication forks and DNA breaks and subsequently activate the checkpoints (Ellison and Stillman 2003). This complex collaborates with ATR in activation of Chk1 and downstream effectors (Weiss, Matsuoka et al. 2002). Further, there is increasing evidence that 9-1-1 is directly involved in DNA repair by stimulating FEN1 (Wang, Brandt et al. 2004). The 9-1-1 orthologs were identified in genetic screens in yeast as proteins that affect sensitivity to DNA damaging agents (Parrilla-Castellar, Arlander et al. 2004). Structural studies predict that the 9-1-1 complex functions as a toroidal sliding clamp in a similar way to the replication clamp, PCNA (Thelen, Venclovas et al. 1999). The 9-1-1 clamp is loaded onto DNA upon damage, and responds by activating signaling pathways that result in DNA repair and maintenance of genomic stability. 9-1-1 is therefore a multifunctional complex that coordinates checkpoint activation with DNA repair. To maintain genomic stability, DNA replication must be completed with high fidelity and any lesion repaired without mutation. Failure to coordinate replication with repair can result in accumulation of lesions leading to a mutagenic phenotype resulting in genomic instability.

The importance of checkpoint proteins in preventing genomic instability and malignant transformation has been well established (Hartwell and Kastan 1994; Maser and DePinho 2002). Loss of the 9-1-1 proteins lead to increased spontaneous chromosomal aberrations and sensitivity to genotoxic agents. Studies utilizing siRNA to down-regulate expression of hHUS1 dramatically increases cellular sensitivity to cisplatin in a lung carcinoma cell line (Kinzel, Hall et al. 2002). Further, targeted inactivation of the murine homolgs of Hus1 or Rad9 results in a significant increase of spontaneous chromosomal aberrations, suggesting accumulation of mutations (Weiss, Enoch et al. 2000; Hopkins, Auerbach et al. 2004). A study analyzing resected NSCLC specimens showed significant over-expression of hRAD9 in 33% of cases analyzed. hRAD9 over-expression also correlated with significant elevation of Ki-67 expression, a marker of cell proliferation (Maniwa, Yoshimura et al. 2005). The authors hypothesized that hRAD9 functions in the tumor to activate cell cycle checkpoints and suppresses apoptosis. Yet due to the strong proliferative cues caused by loss of other cell cycle control regulators, the cells continue to proliferate. The precise role of hRAD9 in malignant transformation remains to be determined. Interestingly, in a study using murine homologs of hRAD9, Rad9 heterozygous mice demonstrated haploinsufficiency on an ATM heterozygous background, as these cells had an increased transformation frequency, compared to the ATM heterozygous cells (Smilenov, Lieberman et al. 2005). Therefore loss of hRAD9 has an additive effect with proteins involved in the DNA damage response pathway to potentiate tumorigenesis.

Identifying novel genetic interactions with the 9-1-1 complex can lead to targets for drug inactivation to selectively kill tumor cells in which these genes are over-expressed. In addition, the method described herein will help elucidate how these proteins function in malignant transformation. Synthetic dosage lethality, which occurs when an over-expressed gene results in lethality in a deletion mutant background, can be used to identify genes and/or pathways. This methodology has been successfully used to screen the yeast gene disruption library with a mutant of DNA topoisomerase I that shifts the catalytic equilibrium of Top1 toward the covalent enzyme-DNA intermediate mimicking the action of the chemotherapeutic agent camptothecin. The screen has been used to find genes already known to affect topoisomerases as well as novel ones which are active in a set of cellular pathways previously not associated with chemotherapy effects. These novel genetic interactions are currently being pursued as possible targets to enhance camptothecin sensitivity. We propose to use the yeast model to screen for novel protein interactions in the 9-1-1 pathway, which is highly conserved between S. cerevisiae and humans. Once we have identified the genes in yeast, we will identify the human homologs and validate our findings in cancer cells over-expressing hRAD9. Our methodology can potentially identify novel therapeutic targets in human cells to treat cancer where 9-1-1 proteins are over-expressed, such as NSCLC.

Experimental Plan and Methodology

Perform a genetic screen (synthetic dosage lethality) to identify genes that are essential in the presence of over-expression of the yeast homologs of human 9-1-1, Ddc1/Mec3/Rad17.

We will over-express individually or together the members of the yeast 9-1-1 complex, Ddc1, Mec3, and Rad17, and screen the yeast gene disruption library to identify essential genes for viability as a result of sensitization from the over-expressed proteins. We will create plasmids that conditionally over-express either Ddc1, Mec3, or Rad17. Using the method of the invention, these plasmids will be individually transferred to the entire viable yeast genome disruption library. This screen will help identify the genes that cause synthetic lethality in the presence of over-expression of yeast 9-1-1. Since Ddc1, Mec3, and Rad17 function as a complex, we will pay particular attention to genes that overlap in the 3 screens. This approach is particularly appealing to identify novel targets for cancer therapy, since the genes identified are non-essential for viability in the presence of normal cellular gene expression but become essential in the presence of deregulated over-expression of the gene of interest. In a significant percentage of NSCLC, hRAD9 is over-expressed (Maniwa, Yoshimura et al. 2005). Thus, identifying the genes that are essential in the presence of over-expressed hRAD9 will provide novel cancer-specific targets. Since the 9-1-1 pathway is highly conserved between S. cerevisiae and humans we expect that down-regulation of these new genes will either kill NSCLC cells or render them sensitive to chemotherapy agents that block replication or cause DNA damage.

Evaluate the effect of siRNA-based inactivation of the human homologs of the genes identified as above in NSCLC cell lines that over-express hRAD9. Specifically, we will look at cell growth and sensitivity to chemotherapeutic agents. These studies will test the efficacy of the genes as potential therapeutic targets.

We propose to mimic the findings from the above yeast screen in both normal and NSCLC cell lines that over-express hRAD9. To test this hypothesis we will use siRNA techniques to knockdown gene expression of the human homologs of the genes identified in the yeast screen (target gene). Human RAD9 will be over-expressed in normal human fibroblasts from an exogenous plasmid in parallel with siRNA to the target gene. We will assay for cell viability and sensitivity to chemotherapeutic agents in these cells in the presence or absence of expression of the target gene. Commonly siRNA molecules do not completely eliminate gene expression hence residual protein may allow cells to survive but significantly increase sensitivity to chemotherapeutic agents. Previous studies have established that siRNA molecules targeting the 9-1-1 genes and murine knockout models of 9-1-1 render cells hypersensitive to agents such as cisplatin and radiation (Weiss, Enoch et al. 2000; Kinzel, Hall et al. 2002; Hopkins, Auerbach et al. 2004). If the target gene expression is significantly reduced by the siRNA molecules, we expect a marked decrease in viability and increased sensitivity to chemotherapeutic agents. We will validate our findings in NSCLC cells that over-express hRAD9, or as controls, NSCLC cells that have normal or low hRAD9. We will assay NSCLC cells in the presence and absence of the target gene and assay cell growth and sensitivity to chemotherapeutic agents. How over-expression of hRAD9 contributes to tumorigenesis remains elusive. A possible hypothesis might be that over-expression of hRAD9 in cancer cells provides an erroneous signal of an active DNA-damage induced checkpoint, thus preventing them from undergoing apoptosis and allowing continued proliferation. Since most tumor cells acquire genomic instability (Lengauer, Kinzler et al. 1998), disruption of 9-1-1 function might be specifically effective in cells that are genetically unstable. The efficacy and specificity of this approach can be assessed in cancer cells that are genetically unstable compared to normal cells. Moreover, recent studies established that tumor cells express markers of activated DNA repair proteins that precede accumulation of genetic instability (Bartkova, Horejsi et al. 2005; Gorgoulis, Vassiliou et al. 2005). It would be interesting to see if we can differentiate effects in early stage hyperplastic cancer cells compared to late stage malignant cancer. In one aspect the invention provides a method to identify targets for a cancer-specific phenotype that are useful as a cancer therapeutic using hRAD9 as a paradigm.

### Example 5

### Transfer of Plasmids Permitting the Regulated Expression of the Srs2 DNA Helicase or Catalytic Inactive Alleles of Srs2

This example illustrates a screen of the gene disruption library with wild-type and catalytic inactive alleles of the helicase Srs2 which acts as an anti-recombinase in yeast. Approximately 25% of the strains from the yeast deletion library have been screened for effects on growth with overexpression with or without 40 Gray of ionizing radiation, a relatively low dose for yeast strains. Approximately 4% of these strains show slow growth in response to copper-induced expression of either the wild-type SRS2 allele or a catalytic inactive srs2 allele. A fraction of these strains only show a phenotype when overexpression of the SRS2 allele is combined with radiation treatment, indicating that certain genetic backgrounds can be sensitized to damage by expression of this antirecombinase helicase.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

### REFERENCES

Arthur, L. (1991). Characterization of a novel eukaryotic topoisomerase (TOP3) in Saccharomyces cerevisiae that affects recombination and gene expression. Genetics & Development, Columbia University College of Physicians & Surgeons, New York, N.Y.
Bailis, J. M. and S. L. Forsburg (2004). “MCM proteins: DNA damage, mutagenesis and repair.” Curr Opin Genet Dev 14(1): 17-21.
Bessler, J. B., J. Z. Torredagger and V. A. Zakian (2001). “The PifIp subfamily of helicases: region-specific DNA helicases?” Trends Cell Biol 11(2): 60-5.
Boeke, J. D., J. Trueheart, G. Natsoulis and G. R. Fink (1987). “5-Fluoroorotic acid as a selective agent in yeast molecular genetics.” Methods in Enzymology 154: 164-75.
Butt, T. R., E. J. Sternberg, J. A. Gorman, P. Clark, D. Hamer, M. Rosenberg and S. T. Crooke (1984). “Copper metallothionein of yeast, structure of the gene, and regulation of expression.” Proc Natl Acad Sci USA 81(11): 3332-6.
Caruthers, J. M. and D. B. McKay (2002). “Helicase structure and mechanism.” Curr Opin Struct Biol 12(1): 123-33.
Cobb, J. A., L. Bjergbaek, K. Shimada, C. Frei and S. M. Gasser (2003). “DNA polymerase stabilization at stalled replication forks requires Mec I and the RecQ helicase Sgs1.” Embo J 22(16): 4325-36.
Denny, W. A. (2004). “Emerging DNA topisomerase inhibitors as anticancer drugs.” Expert Opin Emerg Drugs 9(1): 105-33.
Dutcher, S. K. (1981). “Internuclear transfer of genetic information in kar1-1/KAR1 heterokaryons in Saccharomyces cerevisiae.” Molecular and Cellular Biology 1(3): 245-53.
Erdeniz, N., U. H. Mortensen and R. Rothstein (1997). “Cloning-free PCR-based allele replacement methods.” Genome Res 7(12): 1174-83.
Fiorani, P., R. J. Reid, A. Schepis, H. R. Jacquiau, H. Guo, P. Thimmaiah, P. Benedetti and M. A. Bjornsti (2004). “The deubiquitinating enzyme Doa4p protects cells from DNA topoisomerase I poisons.” J Biol Chem 279(20): 21271-81.
Gangloff, S., J. P. McDonald, C. Bendixen, L. Arthur and R. Rothstein (1994). “The yeast type I topoisomerase Top3 interacts with Sgs1, a DNA helicase homolog: a potential eukaryotic reverse gyrase.” Mol Cell Biol 14(12): 8391-8.
Georgieva, B. and R. Rothstein (2002). “Kar-mediated plasmid transfer between yeast strains: alternative to traditional transformation methods.” Methods Enzymol 350: 278-89.
Hanna, J. S., E. S. Kroll, V. Lundblad and F. A. Spencer (2001). “Saccharomyces cerevisiae CTF18 and CTF4 are required for sister chromatid cohesion.” Mol Cell Biol 21(9): 3144-58.
Hardy, C. D., N. J. Crisona, M. D. Stone and N. R. Cozzarelli (2004). “Disentangling DNA during replication: a tale of two strands.” Philos Trans R Soc Lond B Biol Sci 359(1441): 39-47.
Hickson, I. D. (2003). “RecQ helicases: caretakers of the genome.” Nat Rev Cancer 3(3): 169-78.
Hugerat, Y., F. Spencer, D. Zenvirth and G. Simchen (1994). “A versatile method for efficient YAC transfer between any two strains.” Genomics 22(1): 108-17.
Huh, W. K., J. V. Falvo, L. C. Gerke, A. S. Carroll, R. W. Howson, J. S. Weissman and E. K. O'Shea (2003). “Global analysis of protein localization in budding yeast.” Nature 425(6959): 686-91.
Jorgensen, P., B. Nelson, M. D. Robinson, Y. Chen, B. Andrews, M. Tyers and C. Boone (2002). “High-resolution genetic mapping with ordered arrays of Saccharomyces cerevisiae deletion mutants.” Genetics 162(3): 1091-9.
Kao, H. I., J. Veeraraghavan, P. Polaczek, J. L. Campbell and R. A. Bambara (2004). “On the roles of Saccharomyces cerevisiae Dna2p and Flap endonuclease 1 in Okazaki fragment processing.” J Biol Chem 279(15): 15014-24.
Karin, M., R. Najarian, A. Haslinger, P. Valenzuela, J. Welch and S. Fogel (1984). “Primary structure and transcription of an amplified genetic locus: The CUP1 locus of yeast.” Proc Natl Acad Sci USA 81(2): 337-341.
Kim, R. A. and J. C. Wang (1989). “Function of DNA topoisomerases as replication swivels in Saccharomyces cerevisiae.” Journal of Molecular Biology 208(2): 257-67.
Kim, R. A. and J. C. Wang (1992). “Identification of the yeast TOP3 gene product as a single strand-specific DNA topoisomerase.” Journal of Biological Chemistry 267: 17178-85.
Klein, H. L. (1997). “RDH54, a RAD54 homologue in Saccharomyces cerevisiae, is required for mitotic diploid-specific recombination and repair and for meiosis.” Genetics 147(4): 1533-43.
Klein, H. L. (2001). “Mutations in recombinational repair and in checkpoint control genes suppress the lethal combination of srs2Delta with other DNA repair genes in Saccharomyces cerevisiae.” Genetics 157(2): 557-65.
Labib, K. and J. F. Diffley (2001). “Is the MCM2-7 complex the eukaryotic DNA replication fork helicase?” Curr Opin Genet Dev 11(1): 64-70.
Lee, S. E., A. Pellicioli, A. Malkova, M. Foiani and J. E. Haber (2001). “The Saccharomyces recombination protein Tid1p is required for adaptation from G2/M arrest induced by a double-strand break.” Curr Biol 11(13): 1053-7.
Liu, A. H. and A. Califano (2003). “CASTOR: clustering algorithm for sequence taxonomical organization and relationships.” J Comput Biol 10(1): 21-45.
Measday, V., D. W. Hailey, I. Pot, S. A. Givan, K. M. Hyland, G. Cagney, S. Fields, T. N. Davis and P. Hieter (2002). “Ctf3p, the Mis6 budding yeast homolog, interacts with Mcm22p and Mcm16p at the yeast outer kinetochore.” Genes Dev 16(1): 101-13.
Measday, V. and P. Hieter (2002). “Synthetic dosage lethality.” Methods Enzymol 350: 316-26.
Megonigal, M. D., J. Fertala and M. A. Bjornsti (1997). “Alterations in the catalytic activity of yeast DNA topoisomerase I result in cell cycle arrest and cell death.” J Biol Chem 272(19): 12801-8.
Nitiss, J. and J. C. Wang (1988). “DNA topoisomerase-targeting antitumor drugs can be studied in yeast.” Proceedings of the National Academy of Sciences of the United States of America 85(20): 7501-5.
Orr-Weaver, T. L., J. W. Szostak and R. J. Rothstein (1983). “Genetic applications of yeast transformation with linear and gapped plasmids.” Methods in Enzymology 101: 228-245.
Parsons, A. B., R. L. Brost, H. Ding, Z. Li, C. Zhang, B. Sheikh, G. W. Brown, P. M. Kane, T. R. Hughes and C. Boone (2004). “Integration of chemical-genetic and genetic interaction data links bioactive compounds to cellular target pathways.” Nat Biotechnol 22(1): 62-9.
Pommier, Y., F. Leteurtre, M. R. Fesen, A. Fujimori, R. Bertrand, E. Solary, G. Kohlhagen and K. W. Kohn (1994). “Cellular determinants of sensitivity and resistance to DNA topoisomerase inhibitors.” Cancer Investigation 12(5): 530-42.
Reid, R., M. Lisby and R. Rothstein (2002). “Cloning-free genome alterations in Saccharomyce cerevisiae using adaptamer-mediated PCR.” Methods in Enzymology 350: 258-277.
Reid, R. J., P. Fiorani, M. Sugawara and M. A. Bjornsti (1999). “CDC45 and DPB11 are required for processive DNA replication and resistance to DNA topoisomerase I-mediated DNA damage.” Proc Natl Acad Sci USA 96(20): 11440-5.
Reid, R. J. D., I. Sunjevaric, M. Keddache and R. Rothstein (2002). “Efficient PCR-based gene disruption in Saccharomyces strains using intergenic primers.” Yeast 19: 319-328.
Ronne, H. and R. Rothstein (1988). “Mitotic sectored colonies: Evidence of heteroduplex DNA formation during direct repeat recombination.” Proceedings of the National Academy of Sciences of the United States of America 85: 2696-2700.
Rothstein, R. J. (1983). One-step gene disruption in yeast. Methods in Enzymology. R. Wu, L. Grossman and K. Moldave. New York, Academic Press, Inc. 101: 202-11.
Solinger, J. A., K. Kiianitsa and W. D. Heyer (2002). “Rad54, a Swi2/Snf2-like recombinational repair protein, disassembles Rad51:dsDNA filaments.” Mol Cell 10(5): 1175-88.
Spencer, F., Y. Hugerat, G. Simchen, O. Hurko, C. Connelly and P. Hieter (1994). “Yeast kar1 mutants provide an effective method for YAC transfer to new hosts.” Genomics 22(1): 118-26.
Tong, A. H., M. Evangelista, A. B. Parsons, H. Xu, G. D. Bader, N. Page, M. Robinson, S. Raghibizadeh, C. W. Hogue, H. Bussey, B. Andrews, M. Tyers and C. Boone (2001). “Systematic genetic analysis with ordered arrays of yeast deletion mutants.” Science 294(5550): 2364-8.
Uetz, P., L. Giot, G. Cagney, T. A. Mansfield, R. S. Judson, J. R. Knight, D. Lockshon, V. Narayan, M. Srinivasan, P. Pochart, A. Qureshi-Emili, Y. Li, B. Godwin, D. Conover, T. Kalbfleisch, G. Vijayadamodar, M. Yang, M. Johnston, S. Fields and J. M. Rothberg (2000). “A comprehensive analysis of protein-protein interactions in Saccharomyces cerevisiae.” Nature 403(6770): 623-7.
Ullsperger, C. J., A. V. Vologodskii and N. R. Cozzarelli (1995). Unlinking of DNA by topoisomerases during DNA replication. Nucleic acids and molecular biology. F. Eckstein and D. M. J. Lilley. Berlin, Springer. 9: 115-142.
Vallen, E. A., M. A. Hiller, T. Y. Scherson and M. D. Rose (1992). “Separate domains of KAR1 mediate distinct functions in mitosis and nuclear fusion.” J Cell Biol 117(6): 1277-87.
Walker, J. V., K. C. Nitiss, L. H. Jensen, C. Mayne, T. Hu, P. B. Jensen, M. Sehested, T. Hsieh and J. L. Nitiss (2004). “A mutation in human topoisomerase II alpha whose expression is lethal in DNA repair-deficient yeast cells.” J Biol Chem 279(25): 25947-54.
Wallis, J. W., G. Chrebet, G. Brodsky, M. Rolfe and R. Rothstein (1989). “A hyper-recombination mutation in S. cerevisiae identifies a novel eukaryotic topoisomerase.” Cell 58(2): 409-19.
Wang, J. C. (1994). “DNA topoisomerases as targets of therapeutics: an overview.” Adv Pharmacol: 1-19.
Wang, J. C. (1996). “DNA topoisomerases.” Annu Rev Biochem 65: 635-92.
Winzeler, E. A., D. D. Shoemaker, A. Astromoff, H. Liang, K. Anderson, B. Andre, R. Bangham, R. Benito, J. D. Boeke, H. Bussey, A. M. Chu, C. Connelly, K. Davis, F. Dietrich, S. W. Dow, M. El Bakkoury, F. Foury, S. H. Friend, E. Gentalen, G. Giaever, J. H. Hegemann, T. Jones, M. Laub, H. Liao, R. W. Davis and et al. (1999). “Functional characterization of the S. cerevisiae genome by gene deletion and parallel analysis.” Science 285(5429): 901-6.
Worland, S. T. and J. C. Wang (1989). “Inducible overexpression, purification, and active site mapping of DNA topoisomerase II from the yeast Saccharomyces cerevisiae.” Journal of Biological Chemistry 264(8): 4412-6.
Wyrick, J. J. and R. A. Young (2002). “Deciphering gene expression regulatory networks.” Curr Opin Genet Dev 12(2): 130-6.
Conde, J. and G. R. Fink, Proc. Natl. Acad. Sci. USA 73, 3651 (1976).
Marsh, L. and M. D. Rose, in The molecular and cellular biology of the yeast Sachharomyces Broach, J. R., J. R. Pringle, E. W. Jones, Eds. (Cold Spring Harbor Laboratory Press, Cold Spring Harbor, 1997), vol. III, pp. 827.
Rose, M. D. and G. R. Fink, Cell 48, 1047 (1987).
Sherman, S., G. R. Fink and J. B. Hicks, Methods in Yeast Genetics (Cold Spring Harbor Laboratory, Cold Spring Harbor, N.Y., 1986).
Bartkova, J., Z. Horejsi, et al. (2005). “DNA damage response as a candidate anti-cancer barrier in early human tumorigenesis.” Nature 434(7035): 864-70.
Bartkova, J., J. Lukas, et al. (1997). “Aberrations of the G1- and G1/S-regulating genes in human cancer.” Prog Cell Cycle Res 3: 211-20.
Dollinger (2002). Everyon's guide to cancer therapy.
Ellison, V. and B. Stillman (2003). “Biochemical characterization of DNA damage checkpoint complexes: clamp loader and clamp complexes with specificity for 5′ recessed DNA.” PLoS Biol 1(2): E33.
Gorgoulis, V. G., L. V. Vassiliou, et al. (2005). “Activation of the DNA damage checkpoint and genomic instability in human precancerous lesions.” Nature 434(7035): 907-13.
Hartwell, L. H. and M. B. Kastan (1994). “Cell cycle control and cancer.” Science 266(5192): 1821-8.
Hopkins, K. M., W. Auerbach, et al. (2004). “Deletion of mouse rad9 causes abnormal cellular responses to DNA damage, genomic instability, and embryonic lethality.” Mol Cell Biol 24(16): 7235-48.
Kinzel, B., J. Hall, et al. (2002). “Downregulation of Hus1 by antisense oligonucleotides enhances the sensitivity of human lung carcinoma cells to cisplatin.” Cancer 94(6): 1808-14.
Lengauer, C., K. W. Kinzler, et al. (1998). “Genetic instabilities in human cancers.” Nature 396(6712): 643-9.
Maniwa, Y., M. Yoshimura, et al. (2005). “Accumulation of hRad9 protein in the nuclei of nonsmall cell lung carcinoma cells.” Cancer 103(1): 126-32.
Maser, R. S. and R. A. DePinho (2002). “Connecting chromosomes, crisis, and cancer.” Science 297(5581): 565-9.
Melo, J. and D. Toczyski (2002). “A unified view of the DNA-damage checkpoint.” Curr Opin Cell Biol 14(2): 237-45.
Parrilla-Castellar, E. R., S. J. Arlander, et al. (2004). “Dial 9-1-1 for DNA damage: the Rad9-Hus1-Rad1 (9-1-1) clamp complex.” DNA Repair (Amst) 3(8-9): 1009-14.
Smilenov, L. B., H. B. Lieberman, et al. (2005). “Combined haploinsufficiency for ATM and RAD9 as a factor in cell transformation, apoptosis, and DNA lesion repair dynamics.” Cancer Res 65(3): 933-8.
Thelen, M. P., C. Venclovas, et al. (1999). “A sliding clamp model for the Rad1 family of cell cycle checkpoint proteins.” Cell 96(6): 769-70.
Wang, W., P. Brandt, et al. (2004). “The human Rad9-Rad1-Hus1 checkpoint complex stimulates flap endonuclease 1.” Proc Natl Acad Sci USA 101(48): 16762-7.
Weiss, R. S., T. Enoch, et al. (2000). “Inactivation of mouse Hus1 results in genomic instability and impaired responses to genotoxic stress.” Genes Dev 14(15): 1886-98.
Weiss, R. S., S. Matsuoka, et al. (2002). “Hus1 acts upstream of chk1 in a mammalian DNA damage response pathway.” Curr Biol 12(1): 73-7.
Zhou, B. B. and S. J. Elledge (2000). “The DNA damage response: putting checkpoints in perspective.” Nature 408(6811): 433-9.

What is claimed is:

1. A yeast cell comprising chromosomes I through XVI, wherein at least two chromosomes comprise a nucleic acid sequence encoding a counterselectable marker linked to a centromere.

2. A yeast cell comprising chromosomes I through XVI, wherein each chromosome comprises a nucleic acid sequence encoding a counterselectable marker linked to a centromere.

3. The yeast cell of claim 1 or 2, wherein the nucleic acid sequence further comprises a promoter capable of promoting transcription into the centromere of the chromosome, wherein the promoter is inducible or repressible.

4. The yeast cell of claim 3, wherein the counterselectable marker comprises a URA3 gene and the promoter comprises a GAL1 promoter.

5. The yeast cell of claim 1 or 2, wherein the yeast cell is of the genus Saccharomyces.

6. The yeast cell of claim 3, wherein the yeast cell comprises an extrachromosomal nucleic acid comprising a dominant selectable marker, and wherein the yeast genome lacks the selectable marker.

7. The yeast cell of claim 6, wherein lack of the selectable marker creates a metabolic deficiency or drug sensitivity.

8. The yeast cell of claim 7, wherein the selectable marker comprises LEU2, LYS5, TRP1, HIS3 prototrophic gene, the heterologous drug resistance marker KanMX or any combination thereof.

9. The yeast cell of claim 8, further comprising a mutation in the karyogamy pathway which permits yeast mating while preventing nuclear fusion.

10. A method for transferring extrachromosomal genetic material into a recipient yeast cell, the method comprising:
a) mating a recipient yeast cell to the donor yeast cell of claim 1 or 2, wherein at least one of the cells comprises a mutation in the karyogamy pathway,
b) selecting against the donor cell via the counterselectable marker, and
c) selecting for recipient cells which have acquired the extrachromosomal genetic material.

11. A method for transferring extrachromosomal genetic material into a recipient yeast cell, the method comprising:
a) mating a recipient cell to a donor yeast cell of claim 1 or 2, wherein the donor cell comprises a mutation in the karyogamy pathway,
b) selecting against the donor cell,
c) selecting for recipient cells which have acquired the extrachromosomal genetic material.

12. The method of claim 10 or claim 11, wherein the extrachromosomal genetic material comprises comprises a plasmid, an episomal nucleic acid, a mitochondrial nucleic acid or a virus.

13. The method of claim 10 or 11, wherein the steps are automated.

14. A method for transferring extrachromosomal material into a recipient yeast cell comprising the steps of:
a) mating a recipient cell to the donor yeast cell of claim 1 or 2, wherein at least one of the cells comprises a mutation in the karyogamy pathway,
b) selecting against the donor cell,
c) screening for recipient cells which have acquired the extrachromosomal material.

15. A method for transferring extrachromosomal material into a recipient yeast cell, the method comprising:
a) mating a recipient cell to a donor yeast cell of claim 1 or 2, wherein the donor cell comprises a mutation in the karyogamy pathway,
b) selecting against the donor cell,
c) screening for recipient cells which have acquired the extrachromosomal material.

16. The method of claim 14 or 15, wherein the extrachromosomal material comprises cytoplasm, a protein, a prion, an organelle or any combination thereof.

17. A method for making the yeast cell of claim 2, the method comprising:
a) providing a diploid yeast cell that has one copy of chromosomes I through XVI marked with a counterselectable nucleic acid linked to the centromere, wherein the counterselectable nucleic acid comprises URA3,
b) sporulating the diploid,
c) plating the sporulated diploid to generate an original colony of adjacent haploid cells derived from the spores in an single ascus,
d) identifying an original colony which comprises spores that are uracil auxotrophs resistant to 5-FOA via growth on 5-FOA containing medium,
e) isolating uracil prototrophs spores that are adjacent to the uracil auxotrophs spores, and
f) performing a diagnostic PCR to confirm that all sixteen chromosomes in the uracil prototroph have the nucleic acid sequence which comprises URA3.

18. A method for identifying a nucleic acid sequence that exhibits synthetic dosage lethality in a yeast cell, the method comprising:
a) mating a recipient yeast cell to a donor yeast cell of claim 1 or 2, wherein at least one of the donor and recipient cells comprises a mutation in the karyogamy pathway,
b) selecting against the donor cell,
c) selecting for recipient cells which have acquired genetic material transferred from the donor cell,
d) identifying recipient cells which fail to grow due to failure to acquire the genetic material transferred from the donor cell.

19. The method of claim 18, wherein the donor cell expresses a human homologue of a gene associated with a proliferative disease.

20. The method of claim 19, wherein the proliferative disease comprises cancer.

21. The method of claim 18, wherein the recipient yeast cell comprises a cell from a yeast gene knockout library.

22. A method for transferring a yeast chromosome of interest from a donor yeast cell into a recipient yeast cell, the method comprising:
a) mating a recipient yeast cell to the donor yeast cell of claim 1, wherein at least one of the cells comprises a mutation in the karyogamy pathway,
b) selecting against the donor cell via the counterselectable marker, and
c) selecting for recipient cells which have acquired the yeast chromosome of interest, wherein the yeast chromosome of interest does not contain the counterselectable marker.
